(19) United States
(12) Patent Application Publication
(10) Pub. No.: US 2005/0043374 A1
(43) Pub. Date: Feb. 24, 2005

(54) Aryl substituted oxazolidinones with antibacterial activity

(76) Inventor: Michael Barry Gravestock, Waltham, MA (US)

Correspondence Address: ROPES & GRAY LLP, ONE INTERNATIONAL PLACE, BOSTON, MA, 02110-2624, US

(21) Appl. No.: 10/493,609

(22) PCT Filed: Oct. 23, 2002
(86) PCT No.: PCT/GB02/04796

(30) Foreign Application Priority Data
Oct. 25, 2001 (US) 60330589

(51) Int. Cl. A61K 31/427; A61K 31/422; C07D 417/02; C07D 413/02

(52) U.S. Cl. 514/365; 514/380; 548/200; 548/229

(57) ABSTRACT

Compounds of the formula (I), or a pharmaceutically-acceptable salt, or an in-vivo-hydrolysable ester thereof, 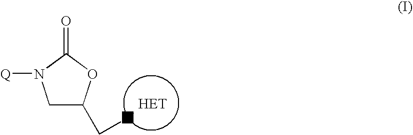 wherein, for example, HET is an N-linked 5-membered, fully or partially unsaturated heterocyclic ring, or HET is an N-linked 6-membered di-hydro-heteroaryl ring;
Q is, for example, Q1 or Q2: 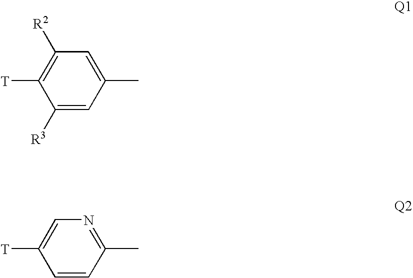
wherein R² and R³ are independently hydrogen or fluoro;
   T is, for example, (TAa1) or (TAa2): 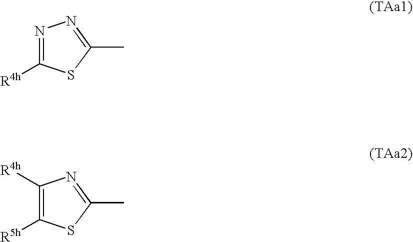
wherein R^{4h} and R^{5h} are independently selected, for example, from hydrogen, halo, trifluoromethyl, cyano, nitro and (1-4C)alkoxy; are useful as antibacterial agents; and processes for their manufacture and pharmaceutical compositions containing them are described.

This application is a national stage filing under 35 U.S.C. 371 of International Application No. PCT/GB02/04796, filed Oct. 23, 2002, which claims priority from Provisional Application No. 60/330,589, filed Oct. 25, 2001, the specification of which is incorporated by reference herein. International Application No. PCT/GB02/04796 was published under PCT Article 21(2) in English.

The present invention relates to antibiotic compounds and in particular to antibiotic compounds containing a substituted oxazolidinone ring. This invention further relates to processes for their preparation, to intermediates useful in their preparation, to their use as therapeutic agents and to pharmaceutical compositions containing them.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, and Streptococci are particularly important because of the development of resistant strains which are both difficult to treat and difficult to eradicate from the hospital environment once established. Examples of such strains are methicillin resistant staphylococcus (MRSA), methicillin resistant coagulase negative staphylococci (MRCNS), penicillin resistant Streptococcus pneumoniae and multiply resistant Enterococcus faecium.

The major clinically effective antibiotic for treatment of such resistant Gram-positive pathogens is vancomycin. Vancomycin is a glycopeptide and is associated with nephrotoxicity and ototoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Certain antibacterial compounds containing an oxazolidinone ring have been described in the art (for example, Walter A. Gregory et al in J. Med. Chem. 1990, 33, 2569-2578 and Chung-Ho Park et al in J. Med. Chem. 1992, 35, 1156-1165). Such antibacterial oxazolidinone compounds with a 5-acetamidomethyl sidechain may be subject to mammalian peptidase metabolism. Furthermore, bacterial resistance to known antibacterial agents may develop, for example, by (i) the evolution of active binding sites in the bacteria rendering a previously active pharmacophore less effective or redundant, (ii) the evolution of means to chemically deactivate a given pharmacophore and/or (iii) the development and/or up-regulation of efflux mechanisms. Therefore, there remains an ongoing need to find new antibacterial agents with a favourable pharmacological profile, in particular for compounds containing new pharmacophores.

We have discovered a new class of antibiotic compounds containing an aryl substituted oxazolidinone ring in which the aryl ring is itself further substituted. These compounds have useful activity against Gram-positive pathogens including MRSA and MRCNS and, in particular, against various strains exhibiting resistance to vancomycin and against E. faecium strains resistant to both aminoglycosides and clinically used β-lactams, but also to fastidious Gram negative strains such as H. influenzae, M. catarrhalis, mycoplasma spp. and chlamydial strains.

Accordingly the present invention provides a compound of the formula (I), or a pharmaceutically-acceptable salt, or an in-vivo-hydrolysable ester thereof, 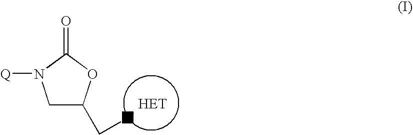
wherein
HET is an N-linked 5-membered, fully or partially unsaturated heterocyclic ring, containing either (i) 1 to 3 further nitrogen heteroatoms or (ii) a further heteroatom selected from O and S together with an optional further nitrogen heteroatom; which ring is optionally substituted on a C atom, other than a C atom adjacent to the linking N atom, by an oxo or thioxo group; and/or which ring is optionally substituted on any available C atom, other than a C atom adjacent to the linking N atom, by a substituent Rs wherein;
Rs is selected from the group:
(Rsa): halogen, (1-4C)alkoxy, (2-4C)alkenyloxy, (2-4C)alkenyl, (2-4C)alkynyl, (3-6C)cycloalkyl, (3-6C)cycloalkenyl, amino, (1-4C)alkylamino, di-(1-4C)alkylamino, (2-4C)alkenylamino, (1-4C)alkylcarbonylamino, (1-4C)alkylthiocarbonylamino, (1-4C)alkyl-OCO—NH—, (1-4C)alkyl-NH—CO—NH—, (1-4C)alkyl-NH—CS—NH—, (1-4C)alkyl-SO₂—NH— or (1-4C)alkyl-S(O)q- (wherein q is 0, 1 or 2);
or Rs is selected from the group
(Rsb): (1-4C)alkyl group which is optionally substituted by one substituent selected from hydroxy, (1-4C)alkoxy, amino, cyano, azido, (2-4C)alkenyloxy, (1-4C)alkylcarbonyl, (1-4C)alkoxycarbonyl, (1-4C)alkylamino, (2-4C)alkenylamino, (1-4C)alkyl-SO₂—NH—, (1-4C)alkylcarbonylamino, (1-4C)alkylthiocarbonylamino, (1-4C)alkyl-OCO—NH—, (1-4C)alkyl-NH—CO—NH—, (1-4C)alkyl-NH—CS—NH—, (1-4C)alkyl-SO₂—NH—, (1-4C)alkyl-S(O)q- (wherein q is 0, 1 or 2), (3-6C)cycloalkyl, (3-6C)cycloalkenyl, or an N-linked 5-membered heteroaryl ring, which ring contains either (i) 1 to 3 further nitrogen heteroatoms or (ii) a further heteroatom selected from O and S together with an optional further nitrogen heteroatom; which ring is optionally substituted on a carbon atom by an oxo or thioxo group; and/or the ring is optionally substituted on a carbon atom by 1 or 2 (1-4C)alkyl groups; and/or on an available nitrogen atom (provided that the ring is not thereby quaternised) by (1-4C)alkyl;
or Rs is selected from a group of formula (Rsc1) to (Rsc3):
(Rsc1): a fully saturated 4-membered monocyclic ring containing 1 or 2 heteroatoms independently selected from O, N and S (optionally oxidised), and linked via a ring nitrogen or carbon atom; or
(Rsc2): a saturated or unsaturated 5-membered monocyclic ring containing 1 heteroatom selected from O, N and S (optionally oxidised), and linked via a ring nitrogen atom if the ring is not thereby quaternised, or a ring carbon atom; or
(Rsc3): a saturated or unsaturated 6- to 8-membered monocyclic ring containing 1 or 2 heteroatoms independently selected from O, N and S (optionally oxidised), and linked via a ring nitrogen atom if the ring is not thereby quaternised, or a ring carbon atom;
wherein said rings in (Rsc1) to (Rsc3) are optionally substituted on an available carbon atom by 1 or 2 substituents independently selected from hydroxy, (1-4C)alkoxy, amino, cyano, azido, (2-4C)alkenyloxy, (1-4C)alkylcarbonyl, (1-4C)alkoxycarbonyl, (1-4C)alkylamino, (2-4C)alkenylamino, (1-4C)alkyl-SO₂—NH—, (1-4C)alkylcarbonylamino, (1-4C)alkylthiocarbonylamino, (1-4C)alkyl-OCO—NH—, (1-4C)alkyl-NH—CO—NH—, (1-4C)alkyl-NH—CS—NH—, (1-4C)alkyl-SO₂—NH—, (1-4C)alkyl-S(O)q- (wherein q is 0, 1 or 2), (3-6C)cycloalkyl or (3-6C)cycloalkenyl;
or Rs is selected from the group
(Rsd): cyano, nitro, azido, formyl, (1-4C)alkylcarbonyl or (1-4C)alkoxycarbonyl; and wherein at each occurrence of an Rs substituent containing an alkyl, alkenyl, alkynyl, cycloalkyl or cycloalkenyl moiety in (Rsa), (Rsb) or (Rsc1) to (Rsc3) each such moiety is optionally further substituted on an available carbon atom with one or more substituents independently selected from F, Cl and Br and/or by one cyano group;
and/or which ring is optionally substituted on an available nitrogen atom (provided that the ring is not thereby quaternised) by (1-4C)alkyl; or
HET is an N-linked 6-membered di-hydro-heteroaryl ring containing up to three nitrogen heteroatoms in total (including the linking heteroatom), which ring is substituted on a suitable C atom, other than a C atom adjacent to the linking N atom, by oxo or thioxo and/or which ring is optionally substituted on any available C atom, other than a C atom adjacent to the linking N atom, by one or two substituents Rs, wherein Rs is as hereinbefore defined, and/or on an available nitrogen atom (provided that the ring is not thereby quaternised) by (1-4C)alkyl; and wherein at each occurrence of alkyl, alkenyl and cycloalkyl HET substituents, each is optionally substituted with one or more substituents independently selected from F, Cl and Br and/or by one cyano group:
Q is selected from Q1 to Q10 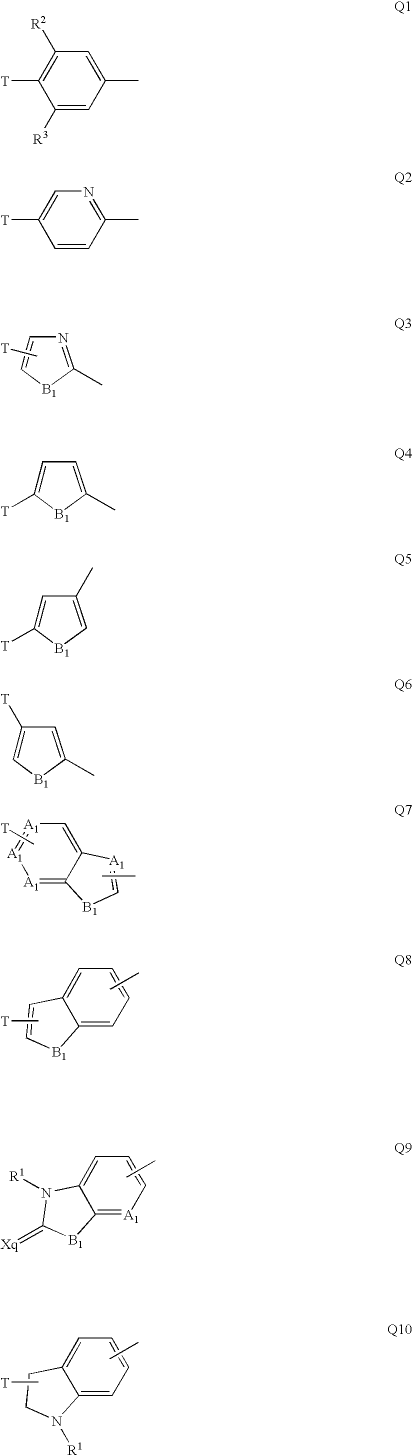
wherein R² and R³ are independently hydrogen or fluoro;
wherein A₁ is carbon or nitrogen; B₁ is O or S (or, in Q9 only, NH); X_{q} is O, S or N—R¹ (wherein R¹ is hydrogen, (1-4C)alkyl or hydroxy-(1-4C)alkyl); and wherein in Q7 each A₁ is independently selected from carbon or nitrogen, with a maximum of 2 nitrogen heteroatoms in the 6-membered ring, and Q7 is linked to T via any of the A₁ atoms (when A₁ is carbon), and linked in the 5-membered ring via the specified carbon atom, or via A₁ when A₁ is carbon; Q8 and Q10 are linked to T via either of the specified carbon atoms in the 5-membered ring, and linked in the benzo-ring via either of the two specified carbon atoms on either side of the linking bond shown; and Q9 is linked via either of the two specified carbon atoms on either side of the linking bond shown;
T is an optionally substituted C-linked (fully unsaturated) 5-membered heteroaryl ring system containing 1, 2 or 3 heteroatoms drawn in combination from O, N, or S, optionally substituted, by one or more substituents independently selected from R^{4h}, R^{5h} and R^{6h} defined hereinafter;
T is preferably selected from the following groups of formula (TAa1) to (TAa6) below (wherein AR1, AR2, AR2a, AR2b, AR3, AR3a, AR3b, AR4, AR4a, CY1 and CY2 are defined hereinbelow); 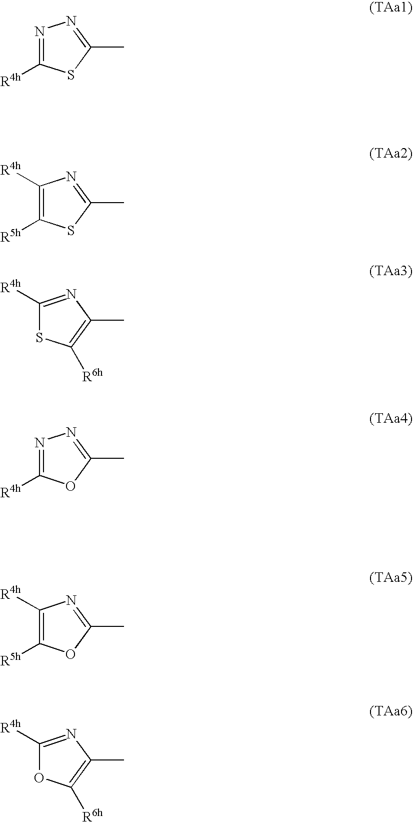
wherein:
R^{6h} is selected from hydrogen, (1-4C)alkyl, (1-4C)alkoxycarbonyl, (1-4C)alkanoyl, carbamoyl and cyano;
R^{4h} and R^{5h} are independently selected from hydrogen, halo, trifluoromethyl, cyano, nitro, (1-4C)alkoxy, (1-4C)alkylS(O)_{q}— (q is 0, 1 or 2), (1-4C)alkanoyl, (1-4C)alkoxycarbonyl, benzyloxy-(1-4C)alkyl, (2-4C)alkanoylamino, —CONRcRv and —NRcRv wherein any (1-4C)alkyl group contained in the preceding values for R^{4h} and R^{5h} is optionally substituted by up to three substituents independently selected from hydroxy (not on C1 of an alkoxy group, and excluding geminal disubstitution), oxo, trifluoromethyl, cyano, nitro, (1-4C)alkoxy, (2-4C)alkanoyloxy, hydroxyimino, (1-4C)alkoxyimino, (1-4C)alkylS(O)_{q}— (q is 0, 1 or 2), (1-4C)alkylSO₂—NRv-, (1-4C)alkoxycarbonyl, —CONRcRv, and —NRcRv (not on C1 of an alkoxy group, and excluding geminal disubstitution); wherein Rv is hydrogen or (1-4C)alkyl and Rc is as hereinafter defined;
R^{4h} and R^{5h} may further be independently selected from (1-4C)alkyl {optionally substituted by up to three substituents independently selected from hydroxy (excluding geminal disubstitution), oxo, trifluoromethyl, cyano, nitro, (1-4C)alkoxy, (2-4C)alkanoyloxy, hydroxyimino, (1-4C)alkoxyimino, (1-4C)alkylS(O)_{q}— (q is 0, 1 or 2), (1-4C)alkylSO₂—NRv-, (1-4C)alkoxycarbonyl, —CONRcRv, and —NRcRv (excluding geminal disubstitution); wherein Rv is hydrogen or (1-4C)alkyl}; Rc is as hereinafter defined; and wherein
any (1-4C)alkyl group contained in the immediately preceding optional substituents (when R^{4h} and R^{5h} are independently (1-4C)alkyl) is itself optionally substituted by up to three substituents independently selected from hydroxy (not on C1 of an alkoxy group, and excluding geminal disubstitution), oxo, trifluoromethyl, cyano, nitro, (1-4C)alkoxy, (2-4C)alkanoyloxy, hydroxyimino, (1-4C)alkoxyimino, (1-4C)alkylS(O)_{q}— (q is 0, 1 or 2), (1-4C)alkylSO₂—NRv-, (1-4C)alkoxycarbonyl, —CONRcRv, and —NRcRv (not on C1 of an alkoxy group, and excluding geminal disubstitution); wherein Rv is hydrogen or (1-4C)alkyl and Rc is as hereinafter defined;
or R^{4h} is selected from one of the groups in (TAaa) to (TAac) below, or (where appropriate) one of R^{4h} and R^{5h} is selected from the above list of R^{4h} and R^{5h} values, and the other is selected from one of the groups in (TAaa) to (TAac) below:
(TAaa) a group of the formula (TAaa1) 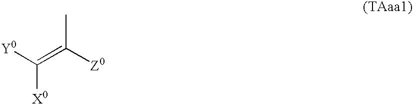
wherein Z⁰ is hydrogen or (1-4C)alkyl;
X⁰ and Y⁰ are independently selected from hydrogen, (1-4C)alkyl, (1-4C)alkoxycarbonyl, halo, cyano, nitro, (1-4C)alkylS(O)_{q}— (q is 0, 1 or 2), RvRwNSO₂—, trifluoromethyl, pentafluoroethyl, (1-4C)alkanoyl and —CONRvRw [wherein Rv is hydrogen or (1-4C)alkyl; Rw is hydrogen or (1-4C)alkyl]; or
one of X⁰ and Y⁰ is selected from the above list of X⁰ and Y⁰ values, and the other is selected from phenyl, phenylcarbonyl, —S(O)_{q}-phenyl (q is 0, 1 or 2), N-(phenyl)carbamoyl, phenylaminosulfonyl, AR2, (AR2)-CO—, (AR2)-S(O)q- (q is 0, 1 or 2), N-(AR2)carbamoyl and (AR2)aminosulfonyl; wherein any phenyl group in (TAaa) may be optionally substituted by up to three substituents independently selected from (1-4C)alkyl, cyano, trifluoromethyl, nitro, halo and (1-4C)alkylsulfonyl;
(TAab) an acetylene of the formula —≡—H or —≡-(1-4C)alkyl;
(TAac) —X¹—Y¹-AR2, —X¹—Y¹-AR2a, —X¹—Y¹-AR2b, —X¹—Y¹-AR3, —X¹—Y¹-AR3a or —X¹—Y¹-AR3b;
wherein X¹ is a direct bond or —CH(OH)— and
Y¹ is —(CH₂)ₘ—, —(CH₂)ₙ—NH—(CH₂)ₘ—, —CO—(CH₂)ₘ—, —CONH—(CH₂)ₘ—, —C(═S)NH—(CH₂)ₘ— or —C(═O)O—(CH₂)ₘ—;
or wherein X¹ is —(CH₂)ₙ— or —CH(Me)-(CH₂)ₘ— and Y¹ is —(CH₂)ₘ—NH—(CH₂)ₘ—, —CO—(CH₂)ₘ—, —CONH—(CH₂)ₘ—, —C(═S)NH—(CH₂)ₘ—, —C(═O)O—(CH₂)ₘ— or —S(O)_{q}—(CH₂)ₘ—;
or wherein X¹ is —CH₂O—, —CH₂NH— or —CH₂N((1-4C)alkyl)- and Y¹ is —CO—(CH₂)ₘ—, —CONH—(CH₂)ₘ— or —C(═S)NH—(CH₂)ₘ—; and additionally Y¹ is —SO₂— when X¹ is —CH₂NH— or —CH₂N((1-4C)alkyl)-, and Y¹ is —(CH₂)ₘ— when X¹ is —CH₂O— or —CH₂N((1-4C)alkyl)-; wherein n is 1, 2 or 3; m is 0, 1, 2 or 3 and q is 0, 1 or 2; and when Y¹ is —(CH₂)ₘ—NH—(CH₂)ₘ— each m is independently selected from 0, 1, 2 or 3;
wherein Rc is selected from groups (Rc1) to (Rc5):
(Rc1) (1-6C)alkyl {optionally substituted by one or more (1-4C)alkanoyl groups (including geminal disubstitution) and/or optionally monosubstituted by cyano, (1-4C)alkoxy, trifluoromethyl, (1-4C)alkoxycarbonyl, phenyl (optionally substituted as for AR1 defined hereinafter), (1-4C)alkylS(O)_{q}— (q is 0, 1 or 2); or, on any but the first carbon atom of the (1-6C)alkyl chain, optionally substituted by one or more groups (including geminal disubstitution) each independently selected from hydroxy and fluoro, and/or optionally monosubstituted by oxo, —NRvRw [wherein Rv is hydrogen or (1-4C)alkyl; Rw is hydrogen or (1-4C)alkyl], (1-6C)alkanoylamino, (1-4C)alkoxycarbonylamino, N-(1-4C)alkyl-N-(1-6C)alkanoylamino, (1-4C)alkylS(O)ₚNH— or (1-4C)alkylS(O)ₚ-((1-4C)alkyl)N— (p is 1 or 2)};
(Rc2) R¹³CO—, R¹³SO₂— or R¹³CS—
wherein R¹³ is selected from (Rc2a) to (Rc2e):
(Rc2a) AR1, AR2, AR2a, AR2b, AR3, AR3a, AR3b, AR4, AR4a, CY1, CY2;
(Rc2b) hydrogen, (1-4C)alkoxycarbonyl, trifluoromethyl, —NRvRw [wherein Rv is hydrogen or (1-4C)alkyl; Rw is hydrogen or (1-4C)alkyl], ethenyl, 2-(1-4C)alkylethenyl, 2-cyanoethenyl, 2-cyano-2-((1-4C)alkyl)ethenyl, 2-nitroethenyl, 2-nitro-2-((1-4C)alkyl)ethenyl, 2-((1-4C)alkylaminocarbonyl)ethenyl, 2-((1-4C)alkoxycarbonyl)ethenyl, 2-(AR1)ethenyl, 2-(AR2)ethenyl, 2-(AR2a)ethenyl;
(Rc2c) (1-10C)alkyl {optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkanoyl, carboxy, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from phosphonate [phosphono, —P(O)(OH)₂, and mono- and di-(1-4C)alkoxy derivatives thereof], phosphinate [—P(OH)₂ and mono- and di-(1-4C)alkoxy derivatives thereof], cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino, (1-4C)alkoxycarbonylamino, N-(1-4C)alkyl-N-(1-6C)alkanoylamino, (1-4C)alkylaminocarbonyl, di((1-4C)alkyl)aminocarbonyl, (1-4C)alkylS(O)ₚNH—, (1-4C)alkylS(O)ₚ-((1-4C)alkyl)N—, fluoro(1-4C)alkylS(O)ₚNH—, fluoro(1-4C)alkylS(O)ₚ((1-4C)alkyl)N—, (1-4C)alkylS(O)_{q}— [the (1-4C)alkyl group of (1-4C)alkylS(O)_{q}— being optionally substituted by one substituent selected from hydroxy, (1-4C)alkoxy, (1-4C)alkanoyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], amino, cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, carboxy, (1-4C)alkylamino, di((1-4C)alkyl)amino, (1-6C)alkanoylamino, (1-4C)alkoxycarbonylamino, N-(1-4C)alkyl-N-(1-6C)alkanoylamino, (1-4C)alkylaminocarbonyl, di((1-4C)alkyl)aminocarbonyl, (1-4C)alkylS(O)ₚNH—, (1-4C)alkylS(O)ₚ-((1-4C)alkyl)N—, (1-4C)alkylS(O)_{q}—, AR1-S(O)_{q}—, AR2-S(O)_{q}—, AR3-S(O)_{q}— and also AR2a, AR2b, AR3a and AR3b versions of AR2 and AR3 containing groups], CY1, CY2, AR1, AR2, AR3, AR1-O—, AR2-O—, AR3-O—, AR1-S(O)_{q}—, AR2-S(O)_{q}—, AR3-S(O)_{q}—, AR1-NH—, AR2-NH—, AR3-NH— (p is 1 or 2 and q is 0, 1 or 2), and also AR2a, AR2b, AR3a and AR3b versions of AR2 and AR3 containing groups};
(Rc2d) R¹⁴C(O)O(1-6C)alkyl wherein R¹⁴ is AR1, AR2, (1-4C)alkylamino (the (1-4C)alkyl group being optionally substituted by (1-4C)alkoxycarbonyl or by carboxy), benzyloxy-(1-4C)alkyl or (1-10C)alkyl {optionally substituted as defined for (Rc2c)};
(Rc2e) R¹⁵O— wherein R¹⁵ is benzyl, (1-6C)alkyl {optionally substituted as defined for (Rc2c)}, CY1, CY2 or AR2b;
(Rc3) hydrogen, cyano, 2-cyanoethenyl, 2-cyano-2-((1-4C)alkyl)ethenyl, 2-((1-4C)alkylaminocarbonyl)ethenyl, 2-((1-4C)alkoxycarbonyl)ethenyl, 2-nitroethenyl, 2-nitro-2-((1-4C)alkyl)ethenyl, 2-(AR1)ethenyl, 2-(AR2)ethenyl, or of the formula
(Rc3a) 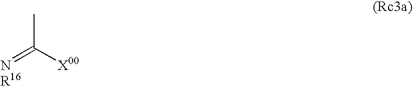
wherein X⁰⁰ is —OR¹⁷, —SR¹⁷, —NHR¹⁷ and —N(R¹⁷)₂;
wherein R¹⁷ is hydrogen (when X⁰⁰ is —NHR¹⁷ and —N(R¹⁷)₂), and R¹⁷ is (1-4C)alkyl, phenyl or AR2 (when X⁰⁰ is —OR¹⁷, —SR¹⁷ and —NHR¹⁷); and R¹⁶ is cyano, nitro, (1-4C)alkylsulfonyl, (4-7C)cycloalkylsulfonyl, phenylsulfonyl, (1-4C)alkanoyl and (1-4C)alkoxycarbonyl;
(Rc4) trityl, AR1, AR2, AR2a, AR2b, AR3, AR3a, AR3b;
(Rc5) RdOC(Re)=CH(C═O)—, RfC(═O)C(═O)—, RgN═C(Rh)C(═O)— or RiNHC(Rj)=CHC(═O)— wherein Rd is (1-6C)alkyl; Re is hydrogen or (1-6C)alkyl, or Rd and Re together form a (3-4C)alkylene chain; Rf is hydrogen, (1-6C)alkyl, hydroxy(1-6C)alkyl, (1-6C)alkoxy(1-6C)alkyl, —NRvRw [wherein Rv is hydrogen or (1-4C)alkyl; Rw is hydrogen or (1-4C)alkyl], (1-6C)alkoxy, (1-6C)alkoxy(1-6C)alkoxy, hydroxy(2-6C)alkoxy, (1-4C)alkylamino(2-6C)alkoxy, di-(1-4C)alkylamino(2-6C)alkoxy; Rg is (1-6C)alkyl, hydroxy or (1-6C)alkoxy; Rh is hydrogen or (1-6C)alkyl; Ri is hydrogen, (1-6C)alkyl, AR1, AR2, AR2a, AR2b and Rj is hydrogen or (1-6C)alkyl; wherein
AR1 is an optionally substituted phenyl or optionally substituted naphthyl;
AR2 is an optionally substituted 5- or 6-membered, fully unsaturated (i.e with the maximum degree of unsaturation) monocyclic heteroaryl ring containing up to four heteroatoms independently selected from O, N and S (but not containing any O—O, O—S or S—S bonds), and linked via a ring carbon atom, or a ring nitrogen atom if the ring is not thereby quaternised;
AR2a is a partially hydrogenated version of AR2 (i.e. AR2 systems retaining some, but not the full, degree of unsaturation), linked via a ring carbon atom or linked via a ring nitrogen atom if the ring is not thereby quaternised;
AR2b is a fully hydrogenated version of AR2 (i.e. AR2 systems having no unsaturation), linked via a ring carbon atom or linked via a ring nitrogen atom;

AR3 is an optionally substituted 8-, 9- or 10-membered, fully unsaturated (i.e with the maximum degree of unsaturation) bicyclic heteroaryl ring containing up to four heteroatoms independently selected from O, N and S (but not containing any O—O, O—S or S—S bonds), and linked via a ring carbon atom in either of the rings comprising the bicyclic system;
AR3a is a partially hydrogenated version of AR3 (i.e. AR3 systems retaining some, but not the full, degree of unsaturation), linked via a ring carbon atom, or linked via a ring nitrogen atom if the ring is not thereby quaternised, in either of the rings comprising the bicyclic system;
AR3b is a fully hydrogenated version of AR3 (i.e. AR3 systems having no unsaturation), linked via a ring carbon atom, or linked via a ring nitrogen atom, in either of the rings comprising the bicyclic system;
AR4 is an optionally substituted 13- or 14-membered, fully unsaturated (i.e with the maximum degree of unsaturation) tricyclic heteroaryl ring containing up to four heteroatoms independently selected from O, N and S (but not containing any O—O, O—S or S—S bonds), and linked via a ring carbon atom in any of the rings comprising the tricyclic system;
AR4a is a partially hydrogenated version of AR4 (i.e. AR4 systems retaining some, but not the full, degree of unsaturation), linked via a ring carbon atom, or linked via a ring nitrogen atom if the ring is not thereby quaternised, in any of the rings comprising the tricyclic system;
CY1 is an optionally substituted cyclobutyl, cyclopentyl or cyclohexyl ring;
CY2 is an optionally substituted cyclopentenyl or cyclohexenyl ring.

In another embodiment, the present invention provides a compound of the formula (I) as hereinbefore described, or a pharmaceutically-acceptable salt, or an in-vivo hydrolysable ester thereof, wherein:
HET is an N-linked 5-membered, fully or partially unsaturated heterocyclic ring, containing either (i) 1 to 3 further nitrogen heteroatoms or (ii) a further heteroatom selected from O and S together with an optional further nitrogen heteroatom; which ring is optionally substituted on a C atom, other than a C atom adjacent to the linking N atom, by an oxo or thioxo group; and/or which ring is optionally substituted on any available C atom, other than a C atom adjacent to the linking N atom, by a substituent selected from (1-4C)alkyl, (2-4C)alkenyl, (3-6C)cycloalkyl, amino, (1-4C)alkylamino, di-(1-4C)alkylamino, (1-4C)alkylthio, (1-4C)alkoxy, (1-4C)alkoxycarbonyl, halogen, cyano and trifluoromethyl and/or on an available nitrogen atom (provided that the ring is not thereby quaternised) by (1-4C)alkyl; or
HET is an N-linked 6-membered di-hydro-heteroaryl ring containing up to three nitrogen heteroatoms in total (including the linking heteroatom), which ring is substituted on a suitable C atom, other than a C atom adjacent to the linking N atom, by oxo or thioxo and/or which ring is optionally substituted on any available C atom, other than a C atom adjacent to the linking N atom, by one or two substituents independently selected from (1-4C)alkyl, (2-4C)alkenyl, (3-6C)cycloalkyl, amino, (1-4C)alkylamino, di-(1-4C)alkylamino, (1-4C)alkylthio, (1-4C)alkoxy, (1-4C)alkoxycarbonyl, halogen, cyano and trifluoromethyl and/or on an available nitrogen atom (provided that the ring is not thereby quaternised) by (1-4C)alkyl; and wherein at each occurrence of alkyl, alkenyl and cycloalkyl HET substituents, each is optionally substituted with one or more F, Cl or CN.
   In this specification, HET as an N-linked 5-membered ring may be a fully or partially unsaturated heterocyclic ring, provided there is some degree of unsaturation in the ring.
   Particular examples of N-linked 5-membered heteroaryl rings containing 2 to 4 heteroatoms independently selected from N, O and S (with no O—O, O—S or S—S bonds) are preferably rings containing 2 to 4 N atoms, in particular pyrazole, imidazole, 1,2,3-triazole (preferably 1,2,3-triazol-1-yl), 1,2,4-triazole (preferably 1,2,4-triazol-1-yl) and tetrazole (preferably tetrazol-2-yl).
   Particular examples of N-linked 6-membered di-hydro-heteroaryl rings containing up to three nitrogen heteroatoms in total (including the linking heteroatom) include di-hydro versions of pyrimidine, pyridazine, pyrazine, 1,2,3-triazine, 1,2,4-triazine, 1,3,5-triazine and pyridine.

It is to be understood that when a value for —X¹— is a two-atom link and is written, for example, as —CH₂NH— it is the left hand part (—CH₂— here) which is bonded to the group of formula (TAa1) to (TAa6) and the right hand part (—NH— here) which is bonded to —Y¹— in the definition in (TAac). Similarly, when —Y¹— is a two-atom link and is written, for example, as —CONH— it is the left hand part of —Y¹— (—CO— here) which is bonded to the right hand part of —X¹—, and the right hand part of —Y¹— (—NH— here) which is bonded to the AR2, AR2a, AR2b, AR3, AR3a or AR3b moiety in the definition in (TAac).

In this specification the term ‘alkyl’ includes straight chained and branched structures. For example, (1-6C)alkyl includes propyl, isopropyl and tert-butyl. However, references to individual alkyl groups such as “propyl” are specific for the straight chained version only, and references to individual branched chain alkyl groups such as “isopropyl” are specific for the branched chain version only. A similar convention applies to other radicals, for example halo(1-4C)alkyl includes 1-bromoethyl and 2-bromoethyl.

There follow particular and suitable values for certain substituents and groups referred to in this specification. These values may be used where appropriate with any of the definitions and embodiments disclosed hereinbefore, or hereinafter.

Examples of (1-4C)alkyl and (1-5C)alkyl include methyl, ethyl, propyl, isopropyl and t-butyl; examples of (1-6C)alkyl include methyl, ethyl, propyl, isopropyl, t-butyl, pentyl and hexyl; examples of (1-10C)alkyl include methyl, ethyl, propyl, isopropyl, pentyl, hexyl, heptyl, octyl and nonyl; examples of (1-4C)alkanoylamino-(1-4C)alkyl include formamidomethyl, acetamidomethyl and acetamidoethyl; examples of hydroxy(1-4C)alkyl and hydroxy(1-6C)alkyl include hydroxymethyl, 1-hydroxyethyl, 2-hydroxyethyl and 3-hydroxypropyl; examples of (1-4C)alkoxycarbonyl include methoxycarbonyl, ethoxycarbonyl and propoxycarbonyl; examples of 2-((1-4C)alkoxycarbonyl)ethenyl include 2-(methoxycarbonyl)ethenyl and 2-(ethoxycarbonyl)ethenyl; examples of 2-cyano-2-((1-4C)alkyl)ethenyl include 2-cyano-2-methylethenyl and 2-cyano-2-ethylethenyl; examples of 2-nitro-2-((1-4C)alkyl)ethenyl include 2-nitro-2-methylethenyl and 2-nitro-2-ethylethenyl; examples of 2-((1-4C)alkylaminocarbonyl)ethenyl include 2-(methylaminocarbonyl)ethenyl and 2-(ethylaminocarbonyl)ethenyl; examples of (2-4C)alkenyl include allyl and vinyl; examples of (2-4C)alkynyl include ethynyl and 2-propynyl; examples of (1-4C)alkanoyl include formyl, acetyl and propionyl; examples of (1-4C)alkoxy include methoxy, ethoxy and propoxy; examples of (1-6C)alkoxy and (1-10C)alkoxy include methoxy, ethoxy, propoxy and pentoxy; examples of (1-4C)alkylthio include methylthio and ethylthio; examples of (1-4C)alkylamino include methylamino, ethylamino and propylamino; examples of di-((1-4C)alkyl)amino include dimethylamino, N-ethyl-N-methylamino, diethylamino, N-methyl-N-propylamino and dipropylamino; examples of halo groups include fluoro, chloro and bromo; examples of (1-4C)alkylsulfonyl include methylsulfonyl and ethylsulfonyl; examples of (1-4C)alkoxy-(1-4C)alkoxy and (1-6C)alkoxy-(1-6C)alkoxy include methoxymethoxy, 2-methoxyethoxy, 2-ethoxyethoxy and 3-methoxypropoxy; examples of (1-4C)alkoxy-(1-4C)alkoxy-(1-4C)alkoxy include 2-(methoxymethoxy)ethoxy, 2-(2-methoxyethoxy)ethoxy; 3-(2-methoxyethoxy)propoxy and 2-(2-ethoxyethoxy)ethoxy; examples of (1-4C)alkylS(O)₂amino include methylsulfonylamino and ethylsulfonylamino; examples of (1-4C)alkanoylamino and (1-6C)alkanoylamino include formamido, acetamido and propionylamino; examples of (1-4C)alkoxycarbonylamino include methoxycarbonylamino and ethoxycarbonylamino; examples of N-(1-4C)alkyl-N-(1-6C)alkanoylamino include N-methylacetamido, N-ethylacetamido and N-methylpropionamido; examples of (1-4C)alkylthiocarbonylamino include MeS—C(═O)—N— and EtS—C(═O)—N—; examples of (1-4C)alkylS(O)ₚNH— wherein p is 1 or 2 include methylsulfinylamino, methylsulfonylamino, ethylsulfinylamino and ethylsulfonylamino; examples of (1-4C)alkylS(O)ₚ((1-4C)alkyl)N— wherein p is 1 or 2 include methylsulfinylmethylamino, methylsulfonylmethylamino, 2-(ethylsulfinyl)ethylamino and 2-(ethylsulfonyl)ethylamino; examples of fluoro(1-4C)alkylS(O)ₚNH— wherein p is 1 or 2 include trifluoromethylsulfinylamino and trifluoromethylsulfonylamino; examples of fluoro(1-4C)alkylS(O)ₚ((1-4C)alkyl)NH— wherein p is 1 or 2 include trifluoromethylsulfinylmethylamino and trifluoromethylsulfonylmethylamino; examples of (1-4C)alkoxy(hydroxy)phosphoryl include methoxy(hydroxy)phosphoryl and ethoxy(hydroxy)phosphoryl; examples of di-(1-4C)alkoxyphosphoryl include di-methoxyphosphoryl, di-ethoxyphosphoryl and ethoxy(methoxy)phosphoryl; examples of (1-4C)alkylS(O)_{q}— wherein q is 0, 1 or 2 include methylthio, ethylthio, methylsulfinyl, ethylsulfinyl, methylsulfonyl and ethylsulfonyl; examples of phenylS(O)_{q} and naphthylS(O)_{q}— wherein q is 0, 1 or 2 are phenylthio, phenylsulfinyl, phenylsulfonyl and naphthylthio, naphthylsulfinyl and naphthylsulfonyl respectively; examples of benzyloxy-(1-4C)alkyl include benzyloxymethyl and benzyloxyethyl; examples of a (3-4C)alkylene chain are trimethylene or tetramethylene; examples of (1-6C)alkoxy-(1-6C)alkyl include methoxymethyl, ethoxymethyl and 2-methoxyethyl; examples of hydroxy-(2-6C)alkoxy include 2-hydroxyethoxy and 3-hydroxypropoxy; examples of (1-4C)alkylamino-(2-6C)alkoxy include 2-methylaminoethoxy and 2-ethylaminoethoxy; examples of di-(1-4C)alkylamino-(2-6C)alkoxy include 2-dimethylaminoethoxy and 2-diethylaminoethoxy; examples of phenyl(1-4C)alkyl include benzyl and phenethyl; examples of (1-4C)alkylcarbamoyl include methylcarbamoyl and ethylcarbamoyl; examples of di((1-4C)alkyl)carbamoyl include di(methyl)carbamoyl and di(ethyl)carbamoyl; examples of hydroxyimino(1-4C)alkyl include hydroxyiminomethyl, 2-(hydroxyimino)ethyl and 1-(hydroxyimino)ethyl; examples of (1-4C)alkoxyimino-(1-4C)alkyl include methoxyiminomethyl, ethoxyiminomethyl, 1-(methoxyimino)ethyl and 2-(methoxyimino)ethyl; examples of halo(1-4C)alkyl include, halomethyl, 1-haloethyl, 2-haloethyl, and 3-halopropyl; examples of nitro(1-4C)alkyl include nitromethyl, 1-nitroethyl, 2-nitroethyl and 3-nitropropyl; examples of amino(1-4C)alkyl include aminomethyl, 1-aminoethyl, 2-aminoethyl and 3-aminopropyl; examples of cyano(1-4C)alkyl include cyanomethyl, 1-cyanoethyl, 2-cyanoethyl and 3-cyanopropyl; examples of (1-4C)alkanesulfonamido include methanesulfonamido and ethanesulfonamido; examples of (1-4C)alkylaminosulfonyl include methylaminosulfonyl and ethylaminosulfonyl; and examples of di-(1-4C)alkylaminosulfonyl include dimethylaminosulfonyl, diethylaminosulfonyl and N-methyl-N-ethylaminosulfonyl; examples of (1-4C)alkanesulfonyloxy include methylsulfonyloxy, ethylsulfonyloxy and propylsulfonyloxy; examples of (1-4C)alkanoyloxy include acetoxy; examples of (1-4C)alkylaminocarbonyl include methylaminocarbonyl and ethylaminocarbonyl; examples of di((1-4C)alkyl)aminocarbonyl include dimethylaminocarbonyl and diethylaminocarbonyl; examples of (3-6C)cycloalkyl and (3-8C)cycloalkyl include cyclopropyl, cyclobutyl, cyclopentyl and cyclohexyl; examples of (4-7C)cycloalkyl include cyclobutyl, cyclopentyl and cyclohexyl; examples of (3-6C)cycloalkenyl include cyclopentenyl and cyclohexenyl; examples of di(N-(1-4C)alkyl)aminomethylimino include dimethylaminomethylimino and diethylaminomethylimino.

Particular values for AR2 include, for example, for those AR2 containing one heteroatom, furan, pyrrole, thiophene; for those AR2 containing one to four N atoms, pyrazole, imidazole, pyridine, pyrimidine, pyrazine, pyridazine, 1,2,3- & 1,2,4-triazole and tetrazole; for those AR2 containing one N and one O atom, oxazole, isoxazole and oxazine; for those AR2 containing one N and one S atom, thiazole and isothiazole; for those AR2 containing two N atoms and one S atom, 1,2,4- and 1,3,4-thiadiazole.

Particular examples of AR2a include, for example, dihydropyrrole (especially 2,5-dihydropyrrol-4-yl) and tetrahydropyridine (especially 1,2,5,6-tetrahydropyrid-4-yl).

Particular examples of AR2b include, for example, tetrahydrofuran, pyrrolidine, morpholine (preferably morpholino), thiomorpholine (preferably thiomorpholino), piperazine (preferably piperazino), imidazoline and piperidine, 1,3-dioxolan4-yl, 1,3-dioxan-4-yl, 1,3-dioxan-5-yl and 1,4-dioxan-2-yl.

Particular values for AR3 include, for example, bicyclic benzo-fused systems containing a 5- or 6-membered heteroaryl ring containing one nitrogen atom and optionally 1-3 further heteroatoms chosen from oxygen, sulfur and nitrogen. Specific examples of such ring systems include, for example, indole, benzofuran, benzothiophene, benzimidazole, benzothiazole, benzisothiazole, benzoxazole, benzisoxazole, quinoline, quinoxaline, quinazoline, phthalazine and cinnoline.

Other particular examples of AR3 include 5/5-, 5/6 and 6/6 bicyclic ring systems containing heteroatoms in both of the rings. Specific examples of such ring systems include, for example, purine and naphthyridine.

Further particular examples of AR3 include bicyclic heteroaryl ring systems with at least one bridgehead nitrogen and optionally a further 1-3 heteroatoms chosen from oxygen, sulfur and nitrogen. Specific examples of such ring systems include, for example, 3H-pyrrolo[1,2-a]pyrrole, pyrrolo[2,1-b]thiazole, 1H-imidazo[1,2-a]pyrrole, 1H-imidazo[1,2-a]imidazole, 1H,3H-pyrrolo[1,2-c]oxazole, 1H-imidazo[1,5-a]pyrrole, pyrrolo[1,2-b]isoxazole, imidazo[5,1-b]thiazole, imidazo[2,1-b]thiazole, indolizine, imidazo[1,2-a]pyridine, imidazo[1,5-a]pyridine, pyrazolo[1,5-a]pyridine, pyrrolo[1,2-b]pyridazine, pyrrolo[1,2-c]pyrimidine, pyrrolo[1,2-a]pyrazine, pyrrolo[1,2-a]pyrimidine, pyrido[2,1-c]-s-triazole, s-triazole[1,5-a]pyridine, imidazo[1,2-c]pyrimidine, imidazo[1,2-a]pyrazine, imidazo[1,2-a]pyrimidine, imidazo[1,5-a]pyrazine, imidazo[1,5-a]pyrimidine, imidazo[1,2-b]-pyridazine, s-triazolo[4,3-a]pyrimidine, imidazo[5,1-b]oxazole and imidazo[2,1-b]oxazole. Other specific examples of such ring systems include, for example, [1H]-pyrrolo[2,1-c]oxazine, [3H]-oxazolo[3,4-a]pyridine, [6H]-pyrrolo[2,1-c]oxazine and pyrido[2,1-c][1,4]oxazine. Other specific examples of 5/5-bicyclic ring systems are imidazooxazole or imidazothiazole, in particular imidazo[5,1-b]thiazole, imidazo[2,1-b]thiazole, imidazo[5,1-b]oxazole or imidazo[2,1-b]oxazole.

Particular examples of AR3a and AR3b include, for example, indoline, 1,3,4,6,9,9a-hexahydropyrido[2,1c][1,4]oxazin-8-yl, 1,2,3,5,8,8a-hexahydroimidazo[1,5a]pyridin-7-yl, 1,5,8,8a-tetrahydrooxazolo[3,4a]pyridin-7-yl, 1,5,6,7,8,8a-hexahydrooxazolo[3,4a]pyridin-7-yl, (7aS)[3H,5H]-1,7a-dihydropyrrolo[1,2c]oxazol-6-yl, (7aS)[5H]-1,2,3,7a-tetrahydropyrrolo[1,2c]imidazol-6-yl, (7aR)[3H,5H]-1,7a-dihydropyrrolo[1,2c]oxazol-6-yl, [3H,5H]-pyrrolo[1,2-c]oxazol-6-yl, [5H]-2,3-dihydropyrrolo[1,2-c]imidazol-6-yl, [3H,5H]-pyrrolo[1,2-c]thiazol-6-yl, [3H,5H]-1,7a-dihydropyrrolo[1,2-c]thiazol-6-yl, [5H]-pyrrolo[1,2-c]imidazol-6-yl, [1H]-3,4,8,8a-tetrahydropyrrolo[2,1-c]oxazin-7-yl, [3H]-1,5,8,8a-tetrahydrooxazolo [3,4-a]pyrid-7-yl, [3H]-5,8-dihydroxazolo[3,4-a]pyrid-7-yl and 5,8-dihydroimidazo [1,5-a]pyrid-7-yl.

Particular values for AR4 include, for example, pyrrolo[a]quinoline, 2,3-pyrroloisoquinoline, pyrrolo[a]isoquinoline, 1H-pyrrolo[1,2-a]benzimidazole, 9H-imidazo[1,2-a]indole, 5H-imidazo[2,1-a]isoindole, 1H-imidazo[3,4-a]indole, imidazo[1,2-a]quinoline, imidazo[2,1-a]isoquinoline, imidazo[1,5-a]quinoline and imidazo[5,1-a]isoquinoline.

The nomenclature used is that found in, for example, “Heterocyclic Compounds (Systems with bridgehead nitrogen), W. L. Mosby (Intercsience Publishers Inc., New York), 1961, Parts 1 and 2.

Where optional substituents are listed such substitution is preferably not geminal disubstitution unless stated otherwise. If not stated elsewhere suitable optional substituents for a particular group are those as stated for similar groups herein.

Suitable substituents on AR1, AR2, AR2a, AR2b, AR3, AR3a, AR3b, AR4, AR4a, CY1 and CY2 are (on an available carbon atom) up to three substituents independently selected from (1-4C)alkyl {optionally substituted by (preferably one) substituents selected independently from hydroxy, trifluoromethyl, (1-4C)alkyl S(O)_{q}— (q is 0, 1 or 2) (this last substituent preferably on AR1 only), (1-4C)alkoxy, (1-4C)alkoxycarbonyl, cyano, nitro,

(1-4C)alkanoylamino, —CONRvRw or —NRvRw}, trifluoromethyl, hydroxy, halo, nitro, cyano, thiol, (1-4C)alkoxy, (1-4C)alkanoyloxy, dimethylaminomethyleneaminocarbonyl, di(N-(1-4C)alkyl)aminomethylimino, carboxy, (1-4C)alkoxycarbonyl, (1-4C)alkanoyl, (1-4C)alkylSO₂amino, (2-4C)alkenyl {optionally substituted by carboxy or (1-4C)alkoxycarbonyl}, (2-4C)alkynyl, (1-4C)alkanoylamino, oxo (═O), thioxo (═S),

(1-4C)alkanoylamino {the (1-4C)alkanoyl group being optionally substituted by hydroxy}, (1-4C)alkyl S(O)_{q}— (q is 0, 1 or 2) {the (1-4C)alkyl group being optionally substituted by one or more groups independently selected from cyano, hydroxy and

(1-4C)alkoxy}, —CONRvRw or —NRvRw [wherein Rv is hydrogen or (1-4C)alkyl; Rw is hydrogen or (1-4C)alkyl].

Further suitable substituents on AR1, AR2, AR2a, AR2b, AR3, AR3a, AR3b, AR4, AR4a, CY1 and CY2 (on an available carbon atom), and also on alkyl groups (unless indicated otherwise) are up to three substituents independently selected from trifluoromethoxy, benzoylamino, benzoyl, phenyl {optionally substituted by up to three substituents independently selected from halo, (1-4C)alkoxy or cyano}, furan, pyrrole, pyrazole, imidazole, triazole, pyrimidine, pyridazine, pyridine, isoxazole, oxazole, isothiazole, thiazole, thiophene, hydroxyimino(1-4C)alkyl, (1-4C)alkoxyimino(1-4C)alkyl, halo-(1-4C)alkyl, (1-4C)alkanesulfonamido, —SO₂NRvRw [wherein Rv is hydrogen or (1-4C)alkyl; Rw is hydrogen or (1-4C)alkyl].

Preferable optional substituents on Ar2b as 1,3-dioxolan4-yl, 1,3-dioxan-4-yl, 1,3-dioxan-5-yl or 1,4-dioxan-2-yl are mono- or disubstitution by substituents independently selected from (1-4C)alkyl (including geminal disubstitution), (1-4C)alkoxy, (1-4C)alkylthio, acetamido, (1-4C)alkanoyl, cyano, trifluoromethyl and phenyl].

Preferable optional substituents on CY1 & CY2 are mono- or disubstitution by substituents independently selected from (1-4C)alkyl (including geminal disubstitution), hydroxy, (1-4C)alkoxy, (1-4C)alkylthio, acetamido, (1-4C)alkanoyl, cyano, and trifluoromethyl.

Suitable substituents on AR2, AR2a, AR2b, AR3, AR3a, AR3b, AR4 and AR4a are (on an available nitrogen atom, where such substitution does not result in quaternization) (1-4C)alkyl, (1-4C)alkanoyl {wherein the (1-4C)alkyl and (1-4C)alkanoyl groups are optionally substituted by (preferably one) substituents independently selected from cyano, hydroxy, nitro, trifluoromethyl, (1-4C)alkyl S(O)_{q}— (q is 0, 1 or 2), (1-4C)alkoxy, (1-4C)alkoxycarbonyl, (1-4C)alkanoylamino, —CONRvRw or —NRvRw [wherein Rv is hydrogen or (1-4C)alkyl; Rw is hydrogen or (1-4C)alkyl]}, (2-4C)alkenyl, (2-4C)alkynyl, (1-4C)alkoxycarbonyl or oxo (to form an N-oxide).

Suitable pharmaceutically-acceptable salts include acid addition salts such as methanesulfonate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

The compounds of the formula (I) may be administered in the form of a pro-drug which is broken down in the human or animal body to give a compound of the formula (I). A prodrug may be used to alter or improve the physical and/or pharmacokinetic profile of the parent compound and can be formed when the parent compound contains a suitable group or substituent which can be derivatised to form a prodrug. Examples of pro-drugs include in-vivo hydrolysable esters of a compound of the formula (I) or a pharmaceutically-acceptable salt thereof.

Various forms of prodrugs are known in the art, for examples see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

An in-vivo hydrolysable ester of a compound of the formula (I) or a pharmaceutically-acceptable salt thereof containing carboxy or hydroxy group is, for example, a pharmaceutically-acceptable ester which is hydrolysed in the human or animal body to produce the parent acid or alcohol.

Suitable pharmaceutically-acceptable esters for carboxy include (1-6C)alkoxymethyl esters for example methoxymethyl, (1-6C)alkanoyloxymethyl esters for example pivaloyloxymethyl, phthalidyl esters, (3-8C)cycloalkoxycarbonyloxy(1-6C)alkyl esters for example 1-cyclohexylcarbonyloxyethyl; 1,3-dioxolan-2-onylmethyl esters for example 5-methyl-1,3-dioxolan-2-ylmethyl; and (1-6C)alkoxycarbonyloxyethyl esters for example 1-methoxycarbonyloxyethyl and may be formed at any carboxy group in the compounds of this invention.

An in-vivo hydrolysable ester of a compound of the formula (I) or a pharmaceutically-acceptable salt thereof containing a hydroxy group or groups includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxymethoxy. A selection of in-vivo hydrolysable ester forming groups for hydroxy include (1-10C)alkanoyl, benzoyl, phenylacetyl and substituted benzoyl and phenylacetyl, (1-10C)alkoxycarbonyl (to give alkyl carbonate esters), di-(1-4C)alkylcarbamoyl and N-(di-(1-4C)alkylaminoethyl)-N-(1-4C)alkylcarbamoyl (to give carbamates), di-(1-4C)alkylaminoacetyl and carboxyacetyl. Examples of substituents on benzoyl and phenylacetyl include chloromethyl or aminomethyl, (1-4C)alkylaminomethyl and di-((1-4C)alkyl)aminomethyl, and morpholino or piperazino linked from a ring nitrogen atom via a methylene linking group to the 3- or 4-position of the benzoyl ring.

Certain suitable in-vivo hydrolysable esters of a compound of the formula (I) are described within the definitions listed in this specification, for example esters described by the definition (Rc2d), and some groups within (Rc2c). Suitable in-vivo hydrolysable esters of a compound of the formula (I) are described as follows. For example, a 1,2-diol may be cyclised to form a cyclic ester of formula (PD1) or a pyrophosphate of formula (PD2): 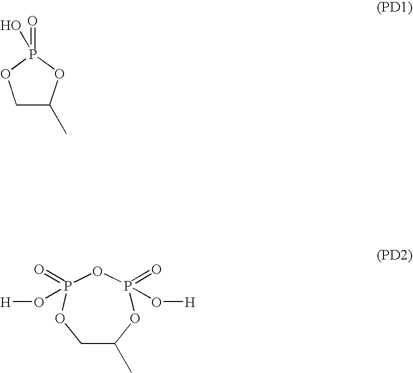
Particularly interesting are such cyclised pro-drugs when the 1,2-diol is on a (1-4C)alkyl chain linked to a carbonyl group in a substituent of formula Rc borne by a nitrogen atom in (TC4). Esters of compounds of formula (I) wherein the HO-function/s in (PD1) and (PD2) are protected by (1-4C)alkyl, phenyl or benzyl are useful intermediates for the preparation of such pro-drugs.

Further in-vivo hydrolysable esters include phosphoramidic esters, and also compounds of formula (I) in which any free hydroxy group independently forms a phosphoryl (npd is 1) or phosphiryl (npd is 0) ester of the formula (PD3)), wherein npd is independently 0 or 1 for each oxo group:

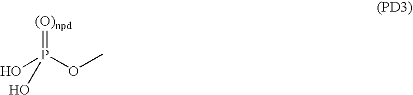

For the avoidance of doubt, phosphono is —P(O)(OH)₂; (1-4C)alkoxy(hydroxy)-phosphoryl is a mono-(1-4C)alkoxy derivative of —O—P(O)(OH)₂; and di-(1-4C)alkoxyphosphoryl is a di-(1-4C)alkoxy derivative of —O—P(O)(OH)₂.

Useful intermediates for the preparation of such esters include compounds containing a group/s of formula (PD3) in which either or both of the —OH groups in (PD3) is independently protected by (1-4C)alkyl (such compounds also being interesting compounds in their own right), phenyl or phenyl-(1-4C)alkyl (such phenyl groups being optionally substituted by 1 or 2 groups independently selected from (1-4C)alkyl, nitro, halo and (1-4C)alkoxy).

Thus, prodrugs containing groups such as (PD1), (PD2) and (PD3) may be prepared by reaction of a compound of formula (I) containing suitable hydroxy group/s with a suitably protected phosphorylating agent (for example, containing a chloro or dialkylamino leaving group), followed by oxidation (if necessary) and deprotection. Prodrugs containing a group such as (PS1) may be obtained by analagous chemistry.

When a compound of formula (I) contains a number of free hydroxy group, those groups not being converted into a prodrug functionality may be protected (for example, using a t-butyl-dimethylsilyl group), and later deprotected. Also, enzymatic methods may be used to selectively phosphorylate or dephosphorylate alcohol functionalities.

Other interesting in-vivo hydrolysable esters include, for example, those in which Rc is defined by, for example, R¹⁴C(O)O(1-6C)alkyl-CO— (wherein R¹⁴ is for example, benzyloxy-(1-4C)alkyl, or phenyl). Suitable substituents on a phenyl group in such esters include, for example, 4-(1-4C)piperazino-(1-4C)alkyl, piperazino-(1-4C)alkyl and morpholino-(1-4C)alkyl.

Where pharmaceutically-acceptable salts of an in-vivo hydrolysable ester may be formed this is achieved by conventional techniques. Thus, for example, compounds containing a group of formula (PD1), (PD2) and/or (PD3) may ionise (partially or fully) to form salts with an appropriate number of counter-ions. Thus, by way of example, if an in-vivo hydrolysable ester prodrug of a compound of formula (I) contains two (PD3) groups, there are four HO—P— functionalities present in the overall molecule, each of which may form an appropriate salt (i.e. the overall molecule may form, for example, a mono-, di-, tri- or tetra-sodium salt).

The compounds of the present invention have a chiral centre at the C-5 position of the isoxazoline ring. The pharmaceutically active enantiomer is of the formula (IA):

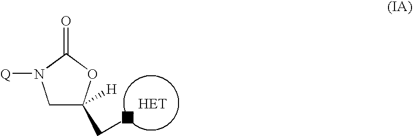

The present invention includes the pure enantiomer depicted above or mixtures of the 5R and 5S enantiomers, for example a racemic mixture. If a mixture of enantiomers is used, a larger amount (depending upon the ratio of the enantiomers) will be required to achieve the same effect as the same weight of the pharmaceutically active enantiomer. For example, the enantiomer depicted above is the 5(R) isomer when HET is 1,2,3- or 1,2,4-triazole or tetrazole.

Furthermore, some compounds of the formula (I) may have other chiral centres. It is to be understood that the invention encompasses all such optical and diastereo-isomers, and racemic mixtures, that possess antibacterial activity. It is well known in the art how to prepare optically-active forms (for example by resolution of the racemic form by recrystallisation techniques, by chiral synthesis, by enzymatic resolution, by biotransformation or by chromatographic separation) and how to determine antibacterial activity as described hereinafter.

Furthermore, some compounds of the formula (I) may exist as cis- and trans-isomers. It is to be understood that the invention encompasses all such isomers, and mixtures thereof, that possess antibacterial activity.

The invention relates to all tautomeric forms of the compounds of the formula (I) that possess antibacterial activity.

It is also to be understood that certain compounds of the formula (I) can exist in solvated as well as unsolvated forms such as, for example, hydrated forms. It is to be understood that the invention encompasses all such solvated forms which possess antibacterial activity.

It is also to be understood that certain compounds of the formula (I) may exhibit polymorphism, and that the invention encompasses all such forms which possess antibacterial activity.

As stated before, we have discovered a range of compounds that have good activity against a broad range of Gram-positive pathogens including organisms known to be resistant to most commonly used antibiotics, together with activity against fastidious Gram negative pathogens such as H.influenzae & M. catarrhalis. They have good physical and/or pharmacokinetic properties in general, and favourable toxicological profiles.

Particularly preferred compounds of the invention comprise a compound of formula (I), or a pharmaceutically-acceptable salt or an in-vivo hydrolysable ester thereof, wherein the substituents Q, HET, T and other substituents mentioned above have values disclosed hereinbefore, or any of the following values (which may be used where appropriate with any of the definitions and embodiments disclosed hereinbefore or hereinafter):

In one embodiment of the invention are provided compounds of formula (I), or a pharmaceutically-acceptable salt or an in-vivo hydrolysable ester thereof, in which Q, HET, T and other substituents mentioned above have the values disclosed hereinbefore and Rs is selected from the group (Rsb).

In one embodiment is provided a compound of formula (I) as defined herein wherein Q is selected from Q1 to Q9. In another embodiment is provided a compound of formula (I) as defined herein wherein Q is Q10.

Preferably Q is selected from Q1, Q2, Q4, Q6 and Q9; especially Q1, Q2 and Q9; more particularly Q1 and Q2; and most preferably Q is Q1.

In one embodiment T is an optionally substituted C-linked (fully unsaturated) 5-membered heteroaryl ring system containing 1, 2 or 3 heteroatoms drawn in combination from O, N, or S, optionally substituted, in a position not adjacent to the linking position, by one or more substituents independently selected from R^{4h}, R^{5h} and 6^{6h} defined herein.

In another embodiment T is selected from the groups of formula (TAa1) to (TAa6) defined herein.

Preferably T is selected from (TAa1 to TAa3). Especially preferred is each of these values of T when present in Q1 and Q2, particularly in Q1.

In one embodiment Rs has values (Rsa) to (Rsc1-3).

In another embodiment Rs has values (Rsd).

Preferable Rs groups are those of (Rsa) and (Rsb).

In one aspect, suitable values of (Rsa) are halo, amino and (2-4C)cycloalkenyl.

In another aspect a suitable value of (Rsd) is cyano.

In (Rsb) the substituted (1-4C)alkyl group is preferably a substituted methyl group.

In one aspect, suitable values for a substituent on a (1-4C)alkyl group in (Rsb) are cyano, azido, halo and (1-4C)alkyl-S(O)q- wherein q=0, particularly wherein the (1-4C)alkyl group is a methyl group.

In (Rsb), when the (1-4C)alkyl group is substituted by a N-linked 5-membered heteroaryl ring it will be appreciated that the ring is aromatic and that when the ring is optionally substituted on an available carbon atom by oxo or thioxo then, when HET contains 1 to 3 further nitrogen heteroatoms, one of the further nitrogen heteroatoms is present as NH or as N-(1-4C)alkyl. Similarly, when the ring is optionally substituted on an available nitrogen atom by (1-4C)alkyl then the ring is substituted on an available carbon atom by oxo or thioxo. Preferred values for the N-linked 5-membered heteroaryl ring as a substituent in (Rsb) are the following rings (HET-P1 to HET-P5):

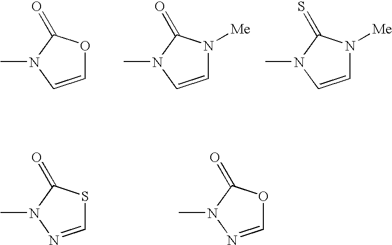

In (Rsc1) to (Rsc3), particular rings are morpholino, tetrahydropyridyl and dihydropyrrolyl.

Preferable (Rs) groups provided by optional F and/or Cl and/or Br and/or one cyano further substituents in (Rsa) and (Rsb) are, for example, Rs as trifluoromethyl, —CHF₂, —CH₂F, —CH₂Cl—CH₂Br, —CH₂CN, —CF₂NH(1-4C)alkyl, —CF₂CH₂OH, —CH₂OCF₃, —CH₂OCHF₂, —CH₂OCH₂F, —NHCF₂CH₃.

In another embodiment, T is selected from TAa1 and TAa2. In a further embodiment, T is TAa1.

Preferably R^{6h} is hydrogen or (1-4C)alkyl, and R^{4h} and R^{5h} are independently selected from hydrogen, cyano, (1-4C)alkoxycarbonyl, —CONRvRw, hydroxy(1-4C)alkyl, NRvRw(1-4C)alkyl, —NRcRv(1-4C)alkyl; wherein Rv is hydrogen or (1-4C)alkyl; Rw is hydrogen or (1-4C)alkyl}; Rc is as hereinbefore defined.

More preferably, R^{5h} and R^{6h} are hydrogen and R^{4h} is selected from cyano, (1-4C)alkoxycarbonyl, —CONRcRv (preferably with Rc as hydrogen or (1-4C)alkyl), hydroxy-(1-4C)alkyl and —NRcRv(1-4C)alkyl; wherein Rv is hydrogen or (1-4C)alkyl and Rc is preferably (Rc2) as hereinbefore defined (especially wherein R¹³ is (Rc2c) as hereinbefore defined).

When R^{4h} and R^{5h} are independently selected from optionally substituted (as defined) (1-4C)alkyl, preferably there are one or two substituents, most especially just one substituent; and when the optional substituent is —CONRcRv or —NRcRv, Rc is preferably hydrogen, (1-4C)alkyl or (1-4C)alkanoyl.

The above preferred values of (TAa) are particularly preferred when present in Q1 or Q2, especially Q1. Most preferable is (TAa1) with preferable R^{4h} substituents as hereinbefore defined.

Preferable values for other substituents (which may be used where appropriate with any of the definitions and embodiments disclosed hereinbefore or hereinafter) are:
(a) In one embodiment HET is a 6-membered heteroaryl as defined herein, and in another embodiment HET is a 5-membered heteroaryl as defined herein.
(b) When HET is a 5-membered heteroaryl as defined herein, preferably HET is 1,2,3-triazole (especially 1,2,3-triazol-1-yl), 1,2,4-triazole (especially 1,2,4-triazol-1-yl) and tetrazole (preferably tetrazol-2-yl).
(c) When HET is a 6-membered heteroaryl as defined herein, preferably HET is a di-hydro version of pyrimidine, pyridazine, pyrazine, 1,2,3-triazine, 1,2,4-triazine, 1,3,5-triazine and pyridine.
(d) In one aspect, preferably HET is unsubstituted. In another aspect HET is substituted as described in any embodiment or aspect described herein.
(e) In one aspect preferably one of R² and R³ is hydrogen and the other fluoro. In another aspect both R² and R³ are fluoro.
(f) Preferably Rc is R¹³CO— and preferably R¹³ is (1-4C)alkoxycarbonyl, hydroxy(1-4C)alkyl, (1-4C)alkyl (optionally substituted by one or two hydroxy groups, or by an (1-4C)alkanoyl group), (1-4C)alkylamino, dimethylamino(1-4C)alkyl, (1-4C)alkoxymethyl, (1-4C)alkanoylmethyl, (1-4C)alkanoyloxy(1-4C)alkyl, (1-5C)alkoxy or 2-cyanoethyl.
(g) More preferably R¹³ is 1,2-dihydroxyethyl, 1,3-dihydroxyprop-2-yl, 1,2,3-trihydroxyprop-1-yl, methoxycarbonyl, hydroxymethyl, methyl, methylamino, dimethylaminomethyl, methoxymethyl, acetoxymethyl, methoxy, methylthio, naphthyl, tert-butoxy or 2-cyanoethyl.
(h) Particularly preferred as R¹³ is 1,2-dihydroxyethyl, 1,3-dihydroxyprop-2-yl or 1,2,3-trihydroxyprop-1-yl.
(i) In another aspect preferably R¹³ is hydrogen, (1-10C)alkyl [optionally substituted by one or more hydroxy] or R¹⁴C(O)O(1-6C)alkyl.

For compounds of formula (I) preferred values for Rc are those in group (Rc2) when present in any of the definitions herein containing Rc.

In the definition of (Rc2c) the AR2a, AR2b, AR3a and AR3b versions of AR2 and AR3 containing groups are preferably excluded.

Especially preferred compounds of the present invention are of the formula (IB): 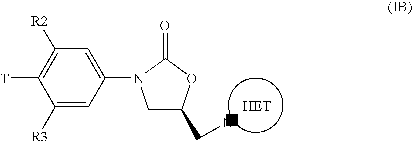
wherein HET is 1,2,3-triazole (especially 1,2,3-triazol-1-yl), 1,2,4-triazole (especially 1,2,4-triazol-1-yl) and tetrazole (preferably tetrazol-2-yl) or HET is a di-hydro version of pyrimidine, pyridazine, pyrazine, 1,2,3-triazine, 1,2,4-triazine, 1,3,5-triazine and pyridine;
R² and R³ are independently hydrogen or fluoro; and
T is selected from (TAa1 to TAa6), or in-vivo hydrolysable esters or pharmaceutically-acceptable salts thereof.

Further especially preferred compounds of the invention are of the formula (IB) wherein HET is 1,2,3-triazole (especially 1,2,3-triazol-1-yl), 1,2,4-triazole (especially 1,2,4-triazol-1-yl) or tetrazole (preferably tetrazol-2-yl;
R² and R³ are independently hydrogen or fluoro;
T is selected from (TAa1 & 2), or in-vivo hydrolysable esters or pharmaceutically-acceptable salts thereof.

In the above aspects and preferred compounds of formula (IB), in (TAa1 to TAa6), preferably R^{5h} and R^{6h} are hydrogen and R^{4h} is selected from cyano, (1-4C)alkoxycarbonyl, —CONRcRv (preferably with Rc as hydrogen or (1-4C)alkyl), hydroxy-(1-4C)alkyl and —NRcRv(1-4C)alkyl; wherein Rv is hydrogen or (1-4C)alkyl and Rc is as defined in (Rc2) and especially R¹³CO— wherein R¹³ is preferably (1-4C)alkoxycarbonyl, hydroxy(1-4C)alkyl, (1-4C)alkyl (optionally substituted by one or two hydroxy groups, or by an (1-4C)alkanoyl group), (1-4C)alkylamino, dimethylamino(1-4C)alkyl, (1-4C)alkoxymethyl, (1-4C)alkanoylmethyl, (1-4C)alkanoyloxy(1-4C)alkyl, (1-5C)alkoxy or 2-cyanoethyl).

In the above aspects and preferred compounds of formula (IB), preferable optional substituents Rs on HET are fluoromethyl, difluoromethyl, trifluoromethyl, chloromethyl, bromomethyl, cyanomethyl, cyano, amino, azido, alkylthioalkyl such as methylthiomethyl, or 2-propynyl.

In all of the above aspects and preferred compounds of formula (IB), in-vivo hydrolysable esters are preferred where appropriate, especially phosphoryl esters (as defined by formula (PD3) with npd as 1).

In all of the above definitions the preferred compounds are as shown in formula (IA). Particular compounds of the present invention include the following Examples, in particular Examples No. 1, and No. 3 and the individual (5R) isomers thereof.

Process Section:

In a further aspect the present invention provides a process for preparing a compound of formula (I) or a pharmaceutically-acceptable salt or an in-vivo hydrolysable ester thereof. It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons).

Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The protecting groups may be removed at any convenient stage in the synthesis using conventional techniques well known in the chemical art.

A compound of the formula (I), or a pharmaceutically-acceptable salt or an in-vivo hydrolysable ester thereof, may be prepared by any process known to be applicable to the preparation of chemically-related compounds. Such processes, when used to prepare a compound of the formula (I), or a pharmaceutically-acceptable salt or an in-vivo hydrolysable ester thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March). The preparation of such starting materials is described within the accompanying non-limiting Examples (in which, for example, 3,5-difluorophenyl, 3-fluorophenyl and (des-fluoro)phenyl containing intermediates may all be prepared by analagous procedures; or by alternative procedures—for example, the preparation of (T group)-(fluoro)phenyl intermediates by reaction of a (fluoro)phenylstannane with, for example, a pyran or (tetrahydro)pyridine compound, may also be prepared by anion chemistry (see, for example, WO97/30995).

Alternatively, necessary starting materials are obtainable by analogous procedures to those illustrated which are within the ordinary skill of an organic chemist. Information on the preparation of necessary starting materials or related compounds (which may be adapted to form necessary starting materials) may also be found in the following Patent and Application Publications, the contents of the relevant process sections of which are hereby incorporated herein by reference: WO99/02525; WO98/54161; WO97/37980; WO97/30981 (& U.S. Pat. No. 5,736,545); WO97/21708 (& U.S. Pat. No. 5,719,154); WO97/10223; WO97/09328; WO96/35691; WO96/23788; WO96/15130; WO96/13502; WO95/25106 (& U.S. Pat. No. 5,668,286); WO95/14684 (& U.S. Pat. No. 5,652,238); WO95/07271 (& U.S. Pat. No. 5,688,792); WO94/13649; WO94/01110; WO93/23384 (& U.S. Pat. No. 5,547,950 & U.S. Pat. No. 5,700,799); WO93/09103 (& U.S. Pat. No. 5,565,571, U.S. Pat. No. 5,654,428, U.S. Pat. No. 5,654,435, U.S. Pat. No. 5,756,732 & U.S. Pat. No. 5,801,246); U.S. Pat. No. 5,231,188; U.S. Pat. No. 5,247,090; U.S. Pat. No. 5,523,403; WO97/27188; WO97/30995; WO97/31917; WO98/01447; WO98/01446; WO99/10342; WO99/10343; WO99/11642; WO99/64416; WO99/64417 and GB99/03299; European Patent Application Nos. 0,359,418 and 0,609,905; 0,693,491 A1 (& U.S. Pat. No. 5,698,574); 0,694,543 A1 (& AU 24985/95); 0,694,544 A1 (& CA 2,154,024); 0,697,412 A1 (& U.S. Pat. No. 5,529,998); 0,738,726 A1 (& AU 50735/96); 0,785,201 A1 (& AU 10123/97); German Patent Application Nos. DE 195 14 313 A1 (& U.S. Pat. No. 5,529,998); DE 196 01 264 A1 (& AU 10098/97); DE 196 01 265 A1 (& AU 10097/97); DE 196 04 223 A1 (& AU 12516/97); DE 196 49 095 A1 (& AU 12517/97).

The following Patent and Application Publications may also provide useful information and the contents of the relevant process sections are hereby incorporated herein by reference: FR 2458547; FR 2500450(& GB 2094299, GB 2141716 & U.S. Pat. No. 4,476,136); DE 2923295 (& GB 2028306, GB 2054575, U.S. Pat. No. 4,287,351, U.S. Pat. No. 4,348,393, U.S. Pat. No. 4,413,001, U.S. Pat. No. 4,435,415 & U.S. Pat. No. 4,526,786), DE 3017499 (& GB 2053196, U.S. Pat. No. 4,346,102 & U.S. Pat. No. 4,372,967); U.S. Pat. No. 4,705,799; European Patent Application Nos. 0,312,000; 0,127,902; 0,184,170; 0,352,781; 0,316,594.

The skilled organic chemist will be able to use and adapt the information contained and referenced within the above references to obtain necessary starting materials.

Thus, the present invention also provides that the compounds of the formulae (I) and pharmaceutically-acceptable salts and in-vivo hydrolysable esters thereof, can be prepared by a process (a) to (i) as follows (wherein the variables are as defined hereinbefore or after unless otherwise stated)
(a) by modifying a substituent in or introducing a substituent into another compound of formula (I); such changes may be usefully made in many positions of compounds of formula (I), for instance a heterocyclyl group linked through nitrogen (optionally substituted on a carbon other than a carbon atom adjacent to the linking nitrogen ring atom) may be converted into another heterocyclyl group linked through nitrogen (optionally substituted on a carbon other than a carbon atom adjacent to the linking nitrogen ring atom) by introduction of a new ring substituent or by refunctionalisation of an existing ring substituent, for instance by modifying the 4-substituent of a 4-substituted 1,2,3-triazol-1-yl group; or for instance such changes may be usefully made in the group Q; for example an alkylthio group may be oxidised to an alkylsulfinyl or alkysulfonyl group, for instance a group R^{4h} that contains an alkylthio group may be oxidized to an alkylsulfinyl or alkylsulfonyl group or for example a group R^{4h} that contains an amino group may be converted into its acylamino derivative in the last step of the preparation of a compound of the formula (I);
   or
(b) by reaction of a compound of formula (II): 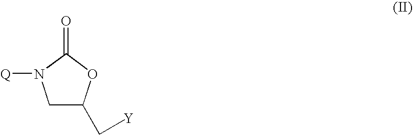
wherein Y is a displaceable group (which may be (i) generated in-situ, for example under Mitsunobu conditions, or (ii) preformed, such as chloro or mesylate) with a compound of the formula (III):
   HET (III)
wherein HET is HET-H free-base form or HET-anion formed from the free base form; or
(c) by reaction of a compound of the formula (IV):
   Q-Z (IV)
wherein Z is an isocyanate, amine or urethane group with an epoxide of the formula (V): 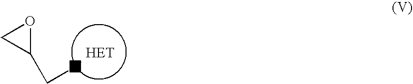
   or
(d) by reaction of a compound of formula (VI): 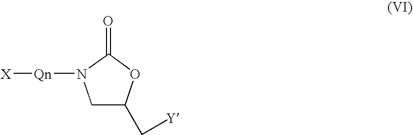
wherein Y′ is a group HET as hereinabove defined, X is a replaceable substituent—such as chloride, bromide, iodide, trifluoromethylsulfonyloxy, trimethylstannyl, trialkoxysilyl, or a boronic acid residue—located at a position substituted by T in any of the aromatic embodiments Q1-Q8 of Qn as hereinabove defined for Q, but with X in place of the substituent T, with a compound of the formula (VII):
   T-X′ (VII)
wherein T-X′ is a five-membered heterocycle with 1-3 heteroatoms drawn in combination from O, N, and S and X′ is a replaceable C-linked substituent—such as chloride, bromide, iodide, trifluoromethylsulfonyloxy, trimethylstannyl, trialkoxysilyl, or a boronic acid residue; wherein the substituents X and X′ are chosen to be complementary pairs of substituents known in the art to be suitable as complementary substrates for coupling reactions catalysed by transition metals such as palladium(0); or
(e) by reaction of a compound of formula (VIII): 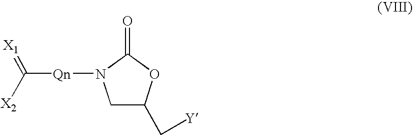
wherein Y′ is a group HET as defined herein above and X1 and X2 here are independently optionally substituted heteroatoms drawn in combination from O, N, and S such that C(X1)X2 constitutes a substituent that is a carboxylic acid derivative substituent located at a position substituted by T in any of the aromatic embodiments Q1-Q10 of Qn as hereinabove defined for Q with a compound of the formula (IX) and X3 and X4 are independently optionally substituted heteroatoms drawn in combination from O, N, and S: 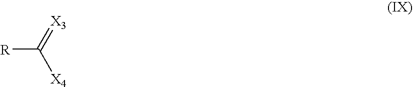
and wherein one of C(X1)X2 and C(X3)X4 constitutes an optionally substituted hydrazide, thiohydrazide, or amidrazone, and the other one of C(X1))X2 and C(X3)X4 constitutes an optionally substituted acylating, thioacylating, or imidoylating agent such that C(X1)X2 and C(X3)X4 may be condensed together to form a 5-membered heterocycle containing 3 heteroatoms drawn in combination from O, N, and S, for instance thiadiazole, by methods well-known in the art; or
(f) by reaction of a compound of formula (X): 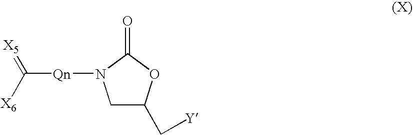
wherein Y′ is a group HET as defined herein above and C(X5)X6 constitutes a substituent located at a position substituted by T in any of the aromatic embodiments Q1-Q8 of Qn as hereinabove defined for Q with a compound of the formula (XI): 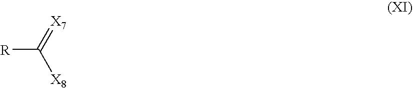
wherein one of C(X5)X6 and C(X7)X8 constitutes an optionally substituted alpha-(leaving-group-substituted)ketone, wherein the leaving group is for example a halo-group or an (alkyl or aryl)-sulfonyloxy-group, and the other one of C(X5)X6 and C(X7)X8 constitutes an optionally substituted amide, thioamide, or amidine, such that C(X5)X6 and C(X7)X8 are groups that may be condensed together to form a 5-membered heterocycle containing 2 heteroatoms drawn in combination from O, N, and S, for instance thiazole, by methods well-known in the art; or
(g) for HET as optionally substituted 1,2,3-triazoles, compounds of the formula (I) may be made by cycloaddition via the azide (wherein e.g. Y in (II) is azide) to acetylenes, or to acetylene equivalents such as optionally substituted cylcohexa-1,4-dienes or optionally substituted ethylenes bearing eliminatable substituents such as arylsulfonyl; or
(h) for HET as 4-substituted 1,2,3-triazole compounds of formula (I) may be made by reacting aminomethylisoxazolines with 1,1-dihaloketone sulfonylhydrazones;
(i) for HET as 4-substituted 1,2,3-triazole compounds of formula (I) may also be made by reacting azidomethyl isoxazolines with terminal alkynes using Cu(1) catalysis;
   and thereafter if necessary: (i) removing any protecting groups; (ii) forming a pharmaceutically-acceptable salt; (iii) forming an in-vivo hydrolysable ester.
(a) Methods for converting substituents into other substituents are known in the art by using standard chemistry (see for example, Comprehensive Organic Functional Group Transformations (Pergamon), Katritzky, Meth-Cohn & Rees); for example: a hydroxy group may be converted into a silyloxy group; an azido or an acylamino or thioacylamino group, for instance an acetamide group (optionally substituted or protected on the amido-nitrogen atom); into an acyloxy group, for instance an acetoxy group; a heterocyclylamino group (optionally substituted or protected on the amino-nitrogen atom), for instance an isoxazol-3-ylamino group or a 1,2,5-thiadiazol-3-ylamino group; a heterocyclyl group linked through nitrogen (optionally substituted on a carbon other than a carbon atom adjacent to the linking nitrogen ring atom), for instance an optionally substituted 1,2,3-triazol-1-yl group; or an amidino group, for instance an 1-(N-cyanoimino)ethylamino group; a hydroxy group may be alkylated to a methoxy group, a hydroxy group may be converted into a halo-group, or into a cyano- group; or into an alkylthio-, an arylthio- or a heteroarylthio-group (see, for example, Tet. Lett., 585, 1972); such conversions of the hydroxy group taking place directly (for instance by acylation or Mitsunobu reaction) or through the intermediacy of one or more derivatives (for instance a mesylate or an azide); moreover, a hydroxy-group may be oxidized to a carbonyl group including a carboxylic acid group. an acyloxy group may be converted into a hydroxy group or into the groups that may be obtained from a hydroxy group (either directly or through the intermediacy of a hydroxy group);
a silyloxy group may be converted into a hydroxy group or into the groups that may be obtained from a hydroxy group (either directly or through the intermediacy of a hydroxy group);
an acylamino group or thioacylamino group may be converted into another acylamino group or thioacylamino group; or into a heterocyclylamino group (optionally substituted or protected on the amino-nitrogen atom);
a carboxylic acid group may be converted into an ester or an amide, an ester may be converted into a carboxylic acid or an amide, and an amide may be converted into an acid or a nitrile; a nitrile may be converted into a carboxylic acid or an amide or an imidate.
an imidate or a nitrile may be converted into a wide range of 5 membered heterocycles such as tetrazoles or 1,2,4-triazoles;
a carbonyl group can be reduced to a hydroxy group and a carboxylic acid group or a derivative thereof can be reduced to a carbonyl group or to a hydroxy group; a carbonyl group may also be converted into a CF₂ group and a carboxylic acid group may be converted into a CF₃ group;
an alkylthio group may be oxidised to an alkylsulfinyl or alkysulfonyl group;
a cyano group may be reduced to an amino group, a nitro group may be reduced to an amino group; a carbonyl group may be converted into a thiocarbonyl group (eg. using Lawsson's reagent) or a bromo group converted to an alkylthio group. It is possible in this way and in closely analogous ways using standard methods well known to skilled organic chemists to interconvert compounds of formula (I).
(b)(i) Reaction (b)(i) (in which Y is initially hydroxy) is performed under Mitsunobu conditions, for example, in the presence of tri-n-butylphosphine and diethyl azodicarboxylate (DEAD) in an organic solvent such as THF, and in the temperature range 0° C.-60° C., but preferably at ambient temperature. Details of Mitsunobu reactions are contained in Tet. Letts., 31, 699, (1990); The Mitsunobu Reaction, D. L. Hughes, Organic Reactions, 1992, Vol. 42, 335-656 and Progress in the Mitsunobu Reaction, D. L. Hughes, Organic Preparations and Procedures International, 1996, Vol. 28, 127-164.
(b)(ii) Reactions (b)(ii) are performed conveniently in the presence of a suitable base such as, for example, an alkali or alkaline earth metal carbonate, alkoxide or hydroxide, for example sodium carbonate or potassium carbonate, or, for example, an organic amine base such as, for example, pyridine, 2,6-lutidine, collidine, 4-dimethylaminopyridine, triethylamine, morpholine or diazabicyclo-[5.4.0]undec-7-ene, the reaction is also preferably carried out in a suitable inert solvent or diluent, for example methylene chloride, acetonitrile, tetrahydrofuran, 1,2-dimethoxyethane, N,N-dimethylformamide, N,N-dimethylacetamide, N-methylpyrrolidin-2-one or dimethylsulfoxide at and at a temperature in the range 25-60° C.

When Y is chloro, the compound of the formula (II) may be formed by reacting a compound of the formula (II) wherein Y is hydroxy (hydroxy compound) with a chlorinating agent. For example, by reacting the hydroxy compound with thionyl chloride, in a temperature range of ambient temperature to reflux, optionally in a chlorinated solvent such as dichloromethane or by reacting the hydroxy compound with carbon tetrachloride/triphenyl phosphine in dichloromethane, in a temperature range of 0° C. to ambient temperature. A compound of the formula (II) wherein Y is chloro or iodo may also be prepared from a compound of the formula (II) wherein Y is mesylate or tosylate, by reacting the latter compound with lithium chloride or lithium iodide and crown ether, in a suitable organic solvent such as THF, in a temperature range of ambient temperature to reflux

When Y is (1-4C)alkanesulfonyloxy or tosylate the compound (II) may be prepared by reacting the hydroxy compound with (1-4C)alkanesulfonyl chloride or tosyl chloride in the presence of a mild base such as triethylamine or pyridine.

When Y is a phosphoryl ester (such as PhO₂—P(O)—O—) or Ph₂-P(O)—O— the compound (II) may be prepared from the hydroxy compound under standard conditions.

If not commercially available, compounds of the formula (III) may be prepared by procedures which are selected from standard chemical techniques, techniques which are analogous to the synthesis of known, structurally similar compounds, or techniques which are analogous to the procedures described in the Examples. For example, standard chemical techniques are as described in Houben Weyl.
(c) by reaction of Q-Z wherein Z is an amine, urethane, or isocyanate with an N-epoxypropyl hetercycle (V). Epoxides of the formula (V) may be prepared from the corresponding N-allylheterocycle of formula (XII):

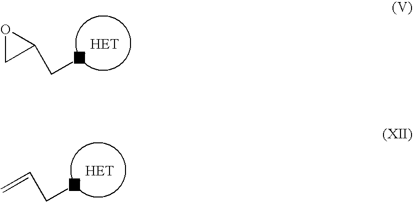

Certain such epoxide and alkene intermediates are novel and are provided as a further feature of the invention. Asymmetric epoxidation may be used to give the desired optical isomer. Furthermore, a similar reaction to reaction (c) may be performed in which Q-Z (wherein Z is a amine group) is reacted with the epoxide (V) (optionally in the presence of an organic base), and the product is reacted with, for example, phosgene to form the oxazolidinone ring.

Alternatively, a precursor of the group HET may be incorporated in place of the group HET in the epoxide of formula (V).

Variations on this process in which the oxirane is replaced by an equivalent reagent X—CH₂CH(O-optionally protected)CH₂HET where X is a displaceable group are also well known in the art.

Such reactions and the preparation of starting materials in within the skill of the ordinary chemist with reference to the above-cited documents disclosing analogous reactions and preparations.

In particular, compounds of the formula (II), and (IV) may be prepared by the skilled man, for example as described in International Patent Application Publication Nos. cited herein, and by analogous processes.

Compounds of the formula (II) wherein Y is hydroxy may be obtained as described in the references cited herein, for example, by reacting a compound Q-Z (IV) where Z is an amine, an isocyanate, or a urethane, especially a compound of the formula (IV, Z=NHCO₂R²¹) with a compound of formula (XIII): 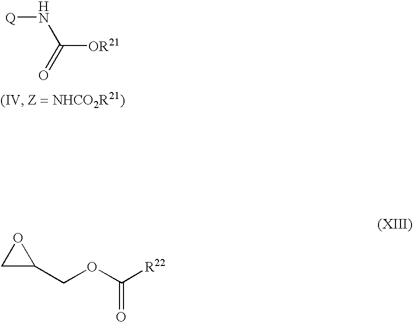
wherein R²¹ is (1-6C)alkyl or benzyl and R²² is (1-4C)alkyl or —S(O)ₙ(1-4C)alkyl where n is 0, 1 or 2. Preferably R²² is (1-4C)alkyl. Compounds of the formula (II), (IV), and (XIII) may be prepared by the skilled man, for example as described in International Patent Application Publication Nos. cited herein, the contents of which are hereby incorporated by reference, and by analogous processes.

Compounds of the formula Q-Z wherein Z is a urethane may be prepared by the skilled chemist, for example by analogous processes to those described in International Patent Application Publication Nos. WO 97/30995 and WO 97/37980. Compounds of the formula Q-Z wherein Z is an isocyanate may be prepared by the skilled chemist, for example by analogous processes to those described in Walter A. Gregory et al in J. Med. Chem. 1990, 33, 2569-2578 and Chung-Ho Park et al in J. Med. Chem. 1992, 35, 1156-1165.
(d) Compounds of formula (II) wherein Y is is HET as hereinabove defined or Y is a group that may be converted to HET, such as hydroxy, may be obtained by coupling together two appropriately substituted fragments to form a carbon-carbon bond in the place of the two substituents X and X′ of (VI) and (VII) respectively. X and X′ may be selected from substituents such as chloro, bromo, iodo, trifluoromethanesulfonyloxy, trialkylstannyl, trialkoxysilyl, or a boronic acid residue provided that the selected substituents X and X′ form a pair of complementary substituents known in the art to be suitable pairs of substituents for transition metal mediated reactions. For instance one of X and X′ prime may be chloro and the other may be trimethylstannyl, as shown in the Scheme.

If not commercially available, the X and X′ substituted fragments used as coupling partners in the transition metal mediated coupling reaction may be prepared by procedures that are selected from standard chemical techniques, techniques that are analogous to the synthesis of known, structurally similar compounds, or techniques which are analogous to the procedures described in the Examples. For example, standard chemical techniques are described in Houben Weyl, Methoden der Organischen Chemie.
The chemistry of process (e) may also be utilised to prepare compounds of formula (II) wherein Y is hydroxy or a group that may be converted into a HET ring, and then process (b) or other suitable chemistry used to prepare compounds of formula (I).
(e) The reaction between thioacylhydrazides and acylating agents to form intermediate acylthioacylhydrazides and subsequently 1,3,4-thiadiazoles is performed under conventional conditions, for instance as indicated in Comprehensive Heterocyclic Chemistry. Suitable acylating agents include acid chlorides and anhydrides. Either of the reaction components may constitute the thioacylhydrazide and the other constitutes the acylating agent, according to synthetic convenience.

The intermediate acylthioacylhydrazides may also be converted into 1,3,4-oxadiazoles. Carboxylic acid derivatives other than thioacylhydrazide are also well known in the art to be substrates for similar reactions leading to other C-linked 5-membered heterocycles containing 3 heteroatoms. For instance, acylhydrazides and amidrazones may be used in place of thioacylhydrazides and thioacylataing agents such as methyl dithioacetate and imidolyating agents such as ethyl acetimidate may be used in place of acylating agents. The products of such reactions are well known in the art. Thus, acylation of acylhydrazides and cyclisation of the resultant diacylhydrazides is known to give 1,3,4-oxadiazoles and acylation amidrazones and cyclisation of the resultant acylamidrazones is known to give 1,2,4-triazoles. Such reactions are described in the literature as indicated in e.g. Comprehensive Heterocyclic Chemistry and are incorporated by reference into the process of method (e). It is further well known that these reactions, for instance the reaction between a (thio)acylating agent and a (thio)hydrazide proceed stepwise to give a cyclic product and that the reactions proceed through intermediates that can themselves be isolated under appropriate reaction conditions. Accordingly this method also includes a process in which an isolable intermediate for instance as di(thio)acylhydrazide is converted into a compound of formula (I) under the conditions already well known for reactions of this type. Moreover, an intermediate in this reaction may be produced under conditions where the cyclization to a compound of formula (I) proceeds spontaneously and without isolation of the intermediate compound, for instance in the conversion of a diacylhydrazide into a thiadiazole under conditions where monothio or dithio diacylhydrazides are formed from the diacylhydrazide and for example Lawesson's reagent. Accordingly this method also includes a process in which an intermediate of the process (e) is formed and spontaneously consumed to give a compound of formula (I) under the conditions already well known for reactions of process (e).

If not commercially available, compounds of the formula (VIII) and (IX) may be prepared by procedures which are selected from standard chemical techniques, techniques which are analogous to the synthesis of known, structurally similar compounds, or techniques which are analogous to the procedures described in the Examples. For example, standard chemical techniques are as described in Houben Weyl.

Certain such thioacylhydrazide and acylating intermediates are novel and are provided as a further feature of the invention (R, Y and Y′ suitable to give compounds of formula (I)). 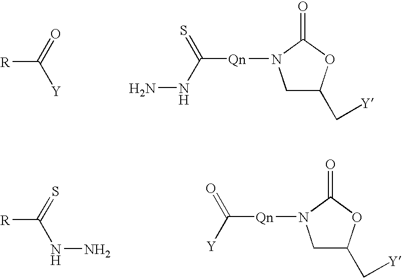
(f) The reaction between thioamides and alpha-haloketones to form thiazoles is performed under conventional conditions, for instance as indicated in Comprehensive Heterocyclic Chemistry, The Chemistry of the Thioamide Group, or Thiazoles (Heterocyclic Chemistry, Weisberger). Suitable halogens include bromine and chlorine. It is well known that other leaving groups may be suitable alternatives to the halogen of an alpha-haloketone, for instance methansulfonyloxy- or hydroxy-ketones or diazoketones may also be used in reaction (f). The regioisomer of thiazole obtained by this method depends on which reaction partner constitutes the thioamide and which reaction partner constitutes the alpha-(leaving-group)substituted-ketone. In the reaction shown in the Scheme the thiazole produced is C2-linked. Carboxylic acid derivatives other than thioamide are also well known in the art to be substates for similar reactions leading to other C-linked 5-membered heterocycles. Such analogous reactions are described in e.g. Comprehensive Heterocyclic Chemistry and are incorporated by reference into the process of method (f). It is further well known that the reaction between an alpha-(leaving-group)substituted-ketone and a thioamide proceeds stepwise to give a 4-hydroxy-4,5-dihydrothiazole as one of the intermediates and that this intermediate can be isolated under appropriate reaction conditions. Accordingly this method also includes a process in which an isolated 4-hydroxy-4,5-dihydrothiazole is converted into a compound of formula (I) under dehydrating conditions already well known for reactions of this type.

If not commercially available, compounds of the formula (X) and (XI) may be prepared by procedures which are selected from standard chemical techniques, techniques which are analogous to the synthesis of known, structurally similar compounds, or techniques which are analogous to the procedures described in the Examples. For example, standard chemical techniques are as described in Houben Weyl. Certain such thioamide and haloketone intermediates are novel and are provided as a further feature of the invention. 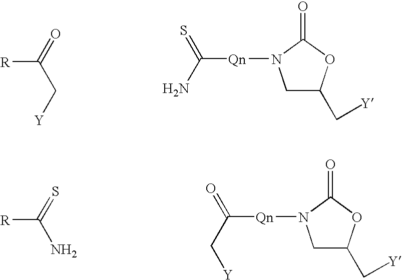
(g) The cycloaddition reaction to form 1,2,3 triazoles from the corresponding azide is performed under conventional conditions. The reaction may use acetylenes or equivalent synthons that react as olefins and then eliminate the elements of a molecule to regenerate a double bond between the carbon atoms of the original olefin. Suitable olefins or their close analogues, which include those able to eliminate the elements of cyclopentadiene, of optionally substituted naphthalenes, of secondary amines, or of sulfinic or sulfenic acids, have been described in the litereature as equivalents to or as synthons for alkynes. The method is illustrated in the Schemes.
(g) 4-Substituted 1,2,3-triazoles may be constructed from a primary amino compound according to the method of Sakai et al. by reacting it with sulfonylhydrazones of 1,1-dihalomethylketones. (see for example Sakai et al., Bull. Chem. Soc. Japan, 1985, 59, 179); as illustrated in the Schemes;
(i) 4-Substituted 1,2,3-triazoles may be constructed from terminal alkynes in a mild and regioselective reaction according to the method of Sharpless.(see V. V. Rostov, L. G. Green, V. V. Folkin, and K. B. Sharpless, Angew. Chem. Int. Ed., 2002, 41, 2596); ); as illustrated in the Schemes; The preparation of suitable alkynes or their close analogues from simpler commercially available acetylenes such as acetylene itself or trimethylsilylacetylene is well-known in the chemical literature;

Compounds of the formula (II) wherein Y is azide may be obtained as described in the references cited herein (particularly in the section proceeding the discussion of protecting groups), for example from the corresponding compounds in which Y is hydroxy or mesylate.

The main synthetic routes are illustrated in the Schemes below (with Q as phenyl, and X, R and A defined with reference to analogous substituents defined elsewhere herein). The compounds of the invention may be prepared by analogous chemistry adapted from the Schemes. The Schemes also show the preparation of 1,2,3-triazoles via the azide (prepared from the relevant hydroxy compound).

The Schemes may be genericised by the skilled man to apply to compounds within the present specification which are not specifically illustrated in the Schemes (for example to HET as a 6-membered ring as defined herein).

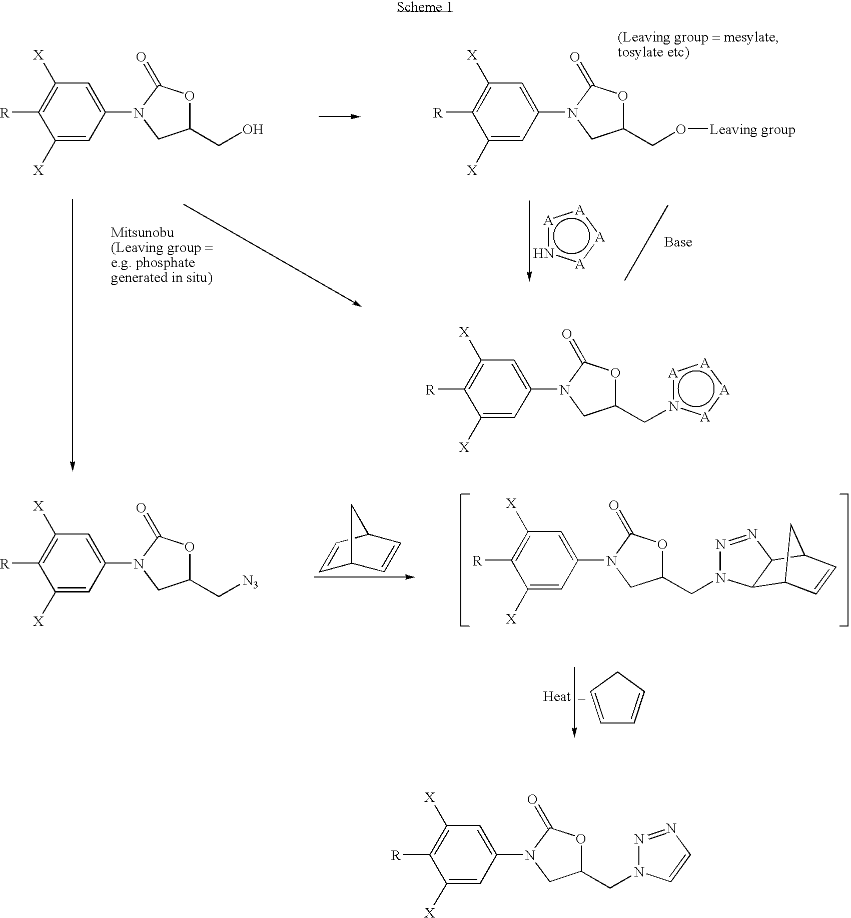

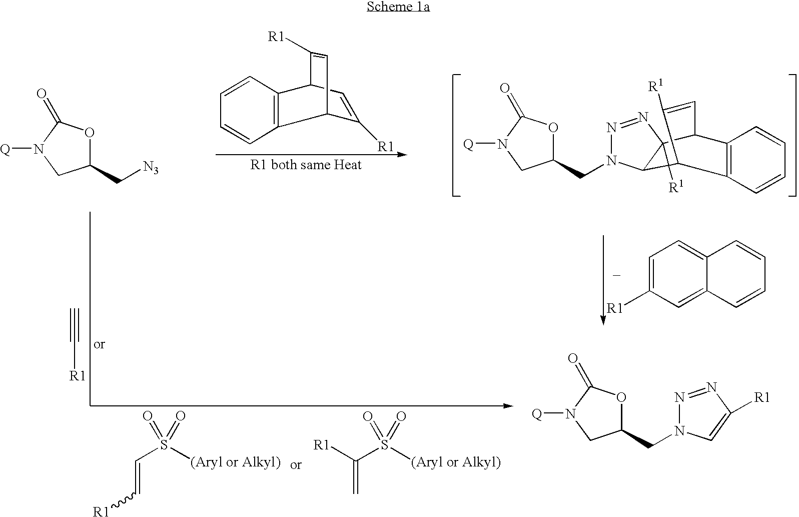

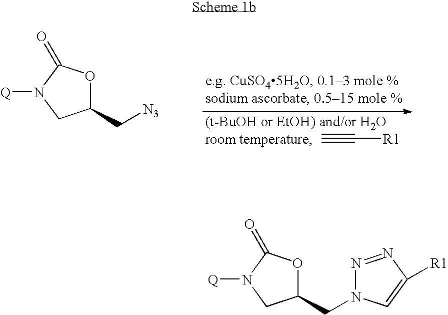

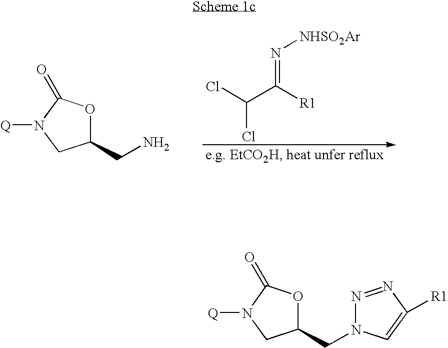

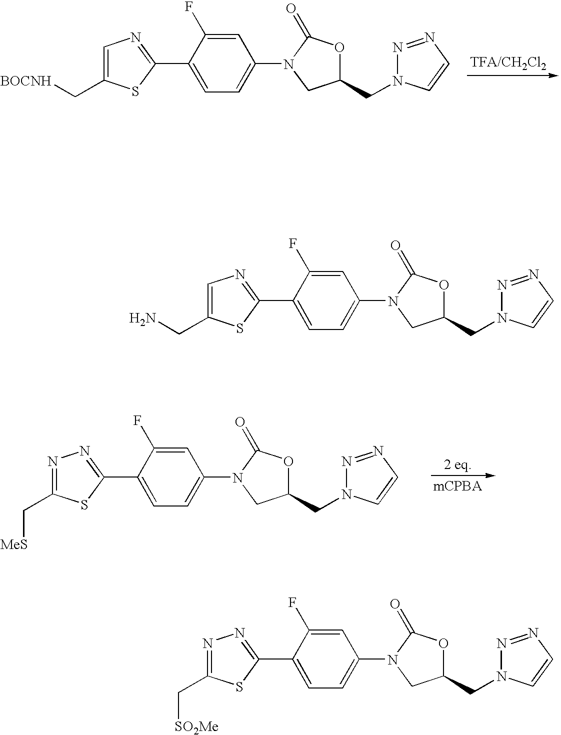

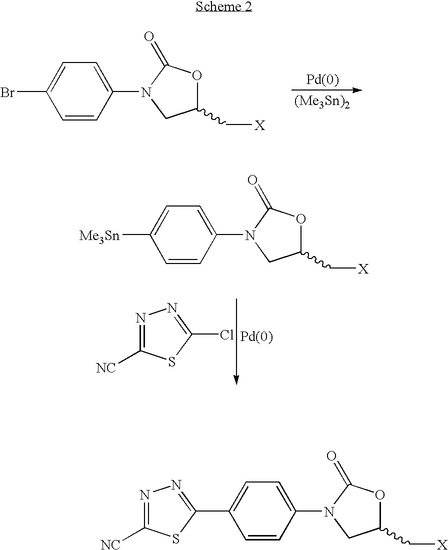

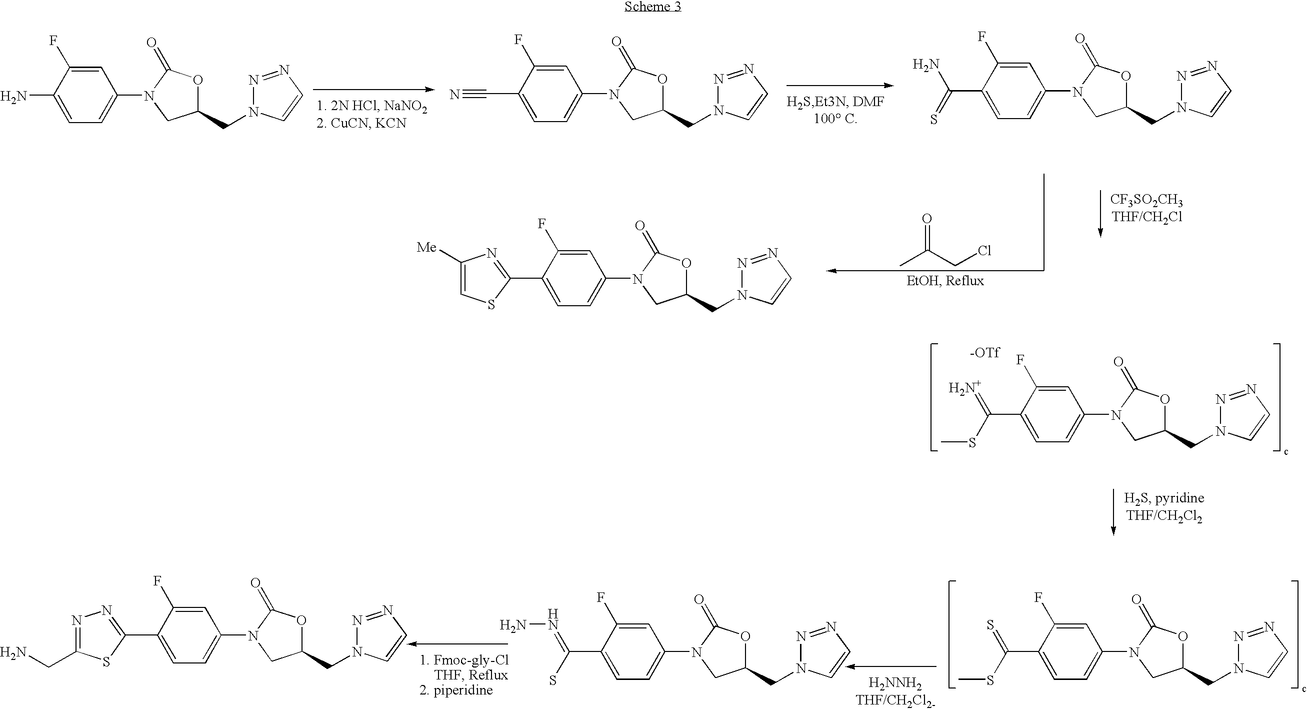

Deprotection, salt formation or in-vivo hydrolysable ester formation may each be provided as a specific final process step.

The N-linked hetereocycle can of course be prepared early in the overall synthesis, and then other functional groups changed.

Where Y is a displaceable group, suitable values for Y are for example, a halogeno or sulfonyloxy group, for example a chloro, bromo, methanesulfonyloxy or toluene-4-sulfonyloxy group.

General guidance on reaction conditions and reagents may be obtained in Advanced Organic Chemistry, 4th Edition, Jerry March (publisher: J. Wiley & Sons), 1992. Necessary starting materials may be obtained by standard procedures of organic chemistry, such as described in this process section, in the Examples section or by analogous procedures within the ordinary skill of an organic chemist. Certain references are also provided which describe the preparation of certain suitable starting materials, for example International Patent Application Publication No. WO 97/37980, the contents of which are incorporated here by reference. Processes analogous to those described in the references may also be used by the ordinary organic chemist to obtain necessary starting materials.

The removal of any protecting groups, the formation of a pharmaceutically-acceptable salt and/or the formation of an in-vivo hydrolysable ester are within the skill of an ordinary organic chemist using standard techniques. Furthermore, details on the these steps, for example the preparation of in-vivo hydrolysable ester prodrugs has been provided in the section above on such esters, and in certain of the following non-limiting Examples.

When an optically active form of a compound of the formula (I) is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the formula (I) is required, it may be obtained by carrying out one of the above procedures using a pure regioisomer as a starting material, or by resolution of a mixture of the regioisomers or intermediates using a standard procedure.

According to a further feature of the invention there is provided a compound of the formula (I), or a pharmaceutically-acceptable salt, or in-vivo hydrolysable ester thereof for use in a method of treatment of the human or animal body by therapy.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt, or in-vivo hydrolysable ester thereof.

The invention also provides a compound of the formula (I), or a pharmaceutically-acceptable salt, or in-vivo hydrolysable ester thereof, for use as a medicament; and the use of a compound of the formula (I) of the present invention, or a pharmaceutically-acceptable salt, or in-vivo hydrolysable ester thereof, in the manufacture of a medicament for use in the production of an antibacterial effect in a warm blooded animal, such as man.

In order to use a compound of the formula (I), an in-vivo hydrolysable ester or a pharmaceutically-acceptable salt thereof, including a pharmaceutically-acceptable salt of an in-vivo hydrolysable ester, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the formula (I), an in-vivo hydrolysable ester or a pharmaceutically-acceptable salt thereof, including a pharmaceutically-acceptable salt of an in-vivo hydrolysable ester, and a pharmaceutically-acceptable diluent or carrier.

The pharmaceutical compositions of this invention may be administered in standard manner for the disease condition that it is desired to treat, for example by oral, rectal or parenteral administration. For these purposes the compounds of this invention may be formulated by means known in the art into the form of, for example, tablets, capsules, aqueous or oily solutions or suspensions, (lipid) emulsions, dispersible powders, suppositories, ointments, creams, aerosols (or sprays), drops and sterile injectable aqueous or oily solutions or suspensions.

In addition to the compounds of the present invention the pharmaceutical composition of this invention may also contain or be co-administered (simultaneously, sequentially or separately) with one or more known drugs selected from other clinically useful antibacterial agents (for example, β-lactams or aminoglycosides) and/or other anti-infective agents (for example, an antifungal triazole or amphotericin). These may include carbapenems, for example meropenem or imipenem, to broaden the therapeutic effectiveness. Compounds of this invention may also contain or be co-administered with bactericidal/permeability-increasing protein (BPI) products or efflux pump inhibitors to improve activity against gram negative bacteria and bacteria resistant to antimicrobial agents.

A suitable pharmaceutical composition of this invention is one suitable for oral administration in unit dosage form, for example a tablet or capsule which contains between 1 mg and 1 g of a compound of this invention, preferably between 100 mg and 1 g of a compound. Especially preferred is a tablet or capsule which contains between 50 mg and 800 mg of a compound of this invention, particularly in the range 100 mg to 500 mg.

In another aspect a pharmaceutical composition of the invention is one suitable for intravenous, subcutaneous or intramuscular injection, for example an injection which contains between 0.1% w/v and 50% w/v (between 1 mg/ml and 500 mg/ml) of a compound of this invention.

Each patient may receive, for example, a daily intravenous, subcutaneous or intramuscular dose of 0.5 mgkg−¹ to 20 mgkg−¹ of a compound of this invention, the composition being administered 1 to 4 times per day. In another embodiment a daily dose of 5 mgkg−¹ to 20 mgkg−¹ of a compound of this invention is administered. The intravenous, subcutaneous and intramuscular dose may be given by means of a bolus injection. Alternatively the intravenous dose may be given by continuous infusion over a period of time. Alternatively each patient may receive a daily oral dose which may be approximately equivalent to the daily parenteral dose, the composition being administered 1 to 4 times per day.

A pharmaceutical composition to be dosed intravenously may contain advantageously (for example to enhance stability) a suitable bactericide, antioxidant or reducing agent, or a suitable sequestering agent.

In the above other, pharmaceutical composition, process, method, use and medicament manufacture features, the alternative and preferred embodiments of the compounds of the invention described herein also apply.

Antibacterial Activity:

The pharmaceutically-acceptable compounds of the present invention are useful antibacterial agents having a good spectrum of activity in-vitro against standard Gram-positive organisms, which are used to screen for activity against pathogenic bacteria. Notably, the pharmaceutically-acceptable compounds of the present invention show activity against enterococci, pneumococci and methicillin resistant strains of S.aureus and coagulase negative staphylococci, together with haemophilus and moraxella strains. The antibacterial spectrum and potency of a particular compound may be determined in a standard test system.

The (antibacterial) properties of the compounds of the invention may also be demonstrated and assessed in-vivo in conventional tests, for example by oral and/or intravenous dosing of a compound to a warm-blooded mammal using standard techniques.

The following results were obtained on a standard in-vitro test system. The activity is described in terms of the minimum inhibitory concentration (MIC) determined by the agar-dilution technique with an inoculum size of 10⁴ CFU/spot. Typically, compounds are active in the range 0.01 to 256 μg/ml.

Staphylococci were tested on agar, using an inoculum of 10⁴ CFU/spot and an incubation temperature of 37° C. for 24 hours—standard test conditions for the expression of methicillin resistance.

Streptococci and enterococci were tested on agar supplemented with 5% defibrinated horse blood, an inoculum of 10⁴ CFU/spot and an incubation temperature of 37° C. in an atmosphere of 5% carbon dioxide for 48 hours—blood is required for the growth of some of the test organisms. Fastidious Gram negative organisms were tested in Mueller-Hinton broth, supplemented with hemin and NAD, grown aerobically for 24 hours at 37° C., and with an innoculum of 5×10⁴ CFU/well.

For example, the following results were obtained for the compound of Example 1:

| Organism | | MIC (μg/ml) |
|---|---|---|
| Staphylococcus aureus: | MSQS | 0.125 |
| | MRQR | 0.25 |
| Streptococcus pneumoniae | | 0.125 |
| Streptococcus pyogenes | | 0.125 |
| Haemophilus influenzae | | 2 |
| Moraxella catarrhalis | | 0.5 |

MSQS = methicillin sensitive and quinolone sensitive
MRQR = methicillin resistant and quinolone resistant

Certain intermediates and/or Reference Examples described hereinafter within the scope of the invention may also possess useful activity, and are provided as a further feature of the invention.

The invention is now illustrated but not limited by the following Examples in which unless otherwise stated:
(i) evaporations were carried out by rotary evaporation in vacuo and work-up procedures were carried out after removal of residual solids by filtration;
(ii) operations were carried out at ambient temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) yields are given for illustration only and are not necessarily the maximum attainable;
(v) the structure of the end-products of the invention were generally confirmed by NMR and mass spectral techniques [proton magnetic resonance spectra were generally determined in DMSO-d₆ unless otherwise stated using a Varian Gemini 2000 spectrometer operating at a field strength of 300 MHz, or a Bruker AM250 spectrometer operating at a field strength of 250 MHz; chemical shifts are reported in parts per million downfield from tetramethysilane as an internal standard (δ scale) and peak multiplicities are shown thus: s, singlet; d, doublet; AB or dd, doublet of doublets; dt, doublet of triplets; dm, doublet of multiplets; t, triplet, m, multiplet; br, broad; fast-atom bombardment (FAB) mass spectral data were generally obtained using a Platform spectrometer (supplied by Micromass) run in electrospray and, where appropriate, either positive ion data or negative ion data were collected];
(vi) each intermediate was purified to the standard required for the subsequent stage and was characterised in sufficient detail to confirm that the assigned structure was correct; purity was assessed by HPLC, TLC, or NMR and identity was determined by infra-red spectroscopy (IR), mass spectroscopy or NMR spectroscopy as appropriate;
(vii) in which the following abbreviations may be used:

DMF is N,N-dimethylformamide; DMA is N,N-dimethylacetamide; TLC is thin layer chromatography; HPLC is high pressure liquid chromatography; MPLC is medium pressure liquid chromatography; DMSO is dimethylsulfoxide; CDCl₃ is deuterated chloroform; MS is mass spectroscopy; ESP is electrospray; EI is electron impact; CI is chemical ionisation; EtOAc is ethyl acetate; MeOH is methanol.

Each of the following Examples comprises an independent aspect of the invention.

### EXAMPLE 1

### (5R)-3-(3-Fluoro-4-(5-cyano-1,3,4-thiadiazol-2-yl)phenyl)-5-(1H-1,2,3-triazol-1-ylmethyl)-1,3-oxazolidin-2-one

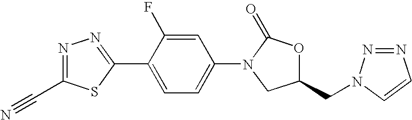

A mixture of (5R)-3-(3-fluoro-4-(trimethylstannyl)phenyl)-5-(1H-1,2,3-triazol-1-ylmethyl)-1,3-oxazolidin-2-one (442 mg, 1.0 mmol), 5-chloro-1,3,4-thiadiazole-2-carbonitrile (151 mg, 1.0 mmol), and triphenylarsine (32 mg, 0.1 mmol) in N-methyl-2-pyrrolidinone (5 ml) under an atmosphere of nitrogen was treated with tris(dibenzylideneacetone)dipalladium(0) (48 mg, 0.05 mmol) and then stirred under an atmosphere of nitrogen for 14 hours at 75° C. The solvent was removed under reduced pressure. A solution of the involatile dark oily residue in ethyl acetate (10 ml) was treated with an aqueous solution of potassium fluoride (2M; 10 ml). The mixture was vortexed for two minutes and then stirred for 15 minutes. The mixture was extracted with ethyl acetate (150 ml) and the extract was dried (MgSO₄). The dried extract was filtered, the filtrate was concentrated under reduced pressure, and the residue was purified by chromatography on silica gel (20 g) (dichloromethane to 2% methanol in dichloromethane gradient) to give the desired product (199 mg). MS (ESP) 372.06 (MH⁺) for C₁₅H₁₀FN₇O₂S. ¹H-NMR (DMSO-d₆) δ: 3.93 (dd, 1H); 4.27 (t, 1H); 4.86 (d, 2H); 5.16 (m, 1H); 7.27 (dd, 1H); 7.44 (m, 1H); 6.98 (t, 1H); 7.78 (s, 1H); 8.16 (s, 1H).

The intermediate for this compound was prepared as follows:
(5R)-5-Azidomethyl-3-(3-fluoro-4-iodophenyl)-13-oxazolidin-2-one

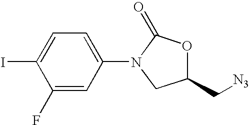

Methanesulfonyl chloride (17.9 ml) was added dropwise to a stirred solution of (5R)-3-(3-fluoro-4-iodophenyl)-5-hydroxymethyl-1,3-oxazolidin-2-one (55.8 g) and triethylamine (46.1 ml) in dry dichloromethane (800 ml) under an atmosphere of dry nitrogen and maintained below room temperature by an ice-bath. The stirred reaction mixture was allowed to warm to room temperature during 3 hours and then washed sequentially with water and brine and then dried (Na₂SO₄). Solvent was removed under reduced pressure to give the intermediate mesylate as a yellow solid (68 g) that was used without further purification.

A stirred solution in DMF (800 ml) of a mixture of the intermediate mesylate (68 g) and sodium azide (32.3 g) was heated at 75° C. overnight. The mixture was allowed to cool to room temperature, diluted with water, and extracted twice with ethyl acetate. The combined extracts were washed sequentially with water and brine, and then dried (Na₂SO₄). Solvent was removed under reduced pressure to give a yellow oil that was purified by column chromatography on silica-gel. Elution with ethyl acetate-hexanes (1:1) gave the product azide as an off-white solid (49 g). The product could be further purified by trituration with ethyl acetate/hexanes.

¹H-NMR (DMSO-d₆) δ: 3.57-3.64 (dd, 1H); 3.70-3.77 (dd, 1H); 3.81-3.87 (dd, 1H); 4.06 (t, 1H); 4.78-4.84 (m, 1H); 7.05-7.09 (ddd, 1H); 7.45 (dd, 1H); 7.68-7.74 (dd, 1H).
(5R)-3-(3-Fluoro-4-iodophenyl)-5-(1H-1,2,3-triazol-1-ylmethyl)-1,3-oxazolidin-2-one

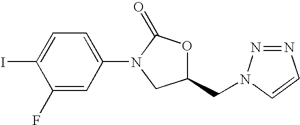

A stirred solution in dioxan (300 ml) of a mixture of the (5R)-5-azidomethyl-3-(3-fluoro4-iodophenyl)-1,3-oxazolidin-2-one (30 g) and bicyclo[2.2.1]heptadiene (30 ml) was heated under reflux overnight. The mixture was allowed to cool to room temperature and then evaporated to dryness under reduced pressure to give a brown solid. The brown solid was purified by column chromatography on silica-gel. Elution with methanol:chloroform (98:2 to 95:5) gave the product triazole as a pale yellow solid (20 g). The product could be further purified by trituration with dichloromethane/hexanes (1:1) to give an off-white solid.

¹H-NMR (DMSO-d₆) δ: 3.86-3.92 (dd, 1H); 4.23 (t, 1H); 4.83 (d, 2H); 5.11-5.19 (m, 1H); 7.12-7.16 (dd, 1H); 7.47-7.51 (dd, 1H); 7.76 (s, 1H); 7.79-7.85 (dd, 1H); 8.16 (s, 1H).
(5R)-3-[3-Fluoro-4-(trimethylstannyl)phenyl]-5-(1H-1,2,3-triazol-1-ylmethyl)-1,3-oxazolidin-2-one

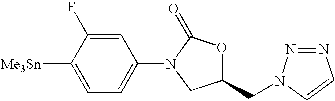

A mixture of (5R)-3-(3-fluoro-4-iodophenyl)-5-(1H-1,2,3-triazol-1-ylmethyl)-1,3-oxazolidin-2-one (5.39 g, 13.9 mmol) and hexamethylditin (5 g, 15.3 mmol) in dioxane (50 ml) under an atmosphere of nitrogen was treated with dichlorobis(triphenylphoshine)palladium (II) (487 mg, 0.69 mmol) and then stirred at 90° C. under an atmosphere of nitrogen for 90 minutes. Silica gel (5 g) was added then the solvent removed under reduced pressure. The residual powder was placed on top of a silica gel column (100 g) and eluted (1% methanol in dichloromethane to 2.5% methanol in dichloromethane gradient) to give the desired product (4.545 g).

MS (ESP) 423, 425, 427 (MH⁺) for C₁₅H₁₉FN₄O₂Sn. ¹H-NMR (DMSO-d₆) δ: 0.32 (s, 9H); 3.90 (dd, 1H); 4.25 (t, 1H); 4.85 (d, 2H); 5.16 (m, 1H); 7.26 (dd, 1H); 7.33 (dd, 1H); 7.41 (dd, 1H); 7.78 (s, 1H); 8.18 (s, 1H).

The preparation of 5-chloro-[1,3,4]thiadiazole-2-carbonitrile is described in
Gadwood, Robert C.; Barbachyn, Michael Robert; Toops, Dana Scott; Smith, Herman Walden; Vaillancourt, Valerie Ann. Preparation of azolylpiperazinylphenyloxazolidinones as antimicrobials. U.S. (1998), 34 pp. CODEN: USXXAM U.S. Pat. No. 5,736,545 A 19980407 CAN 128:270612 AN 1998:219349 CAPLUS
Gadwood, Robert C.; Barbachyn, Michael R.; Toops, Dana S.; Smith, Herman W.; Vaillancourt, Valerie A. Preparation of 3-[4-(4-azolyl-1-piperazinyl)phenyl]oxazolidin-2-ones as bactericides. PCT Int. Appl. (1997), 79 pp. CODEN: PIXXD2 WO 9730981 A1 19970828 CAN 127:278210 AN 1997:579705 CAPLUS

### EXAMPLE 2

### (5R)-3-(3-Fluoro-4-(5-ethoxycarbonyl-1,3,4-thiadiazol-2-yl)phenyl)-5-(1H-1,2,3-triazol-1-ylmethyl)-1,3-oxazolidin-2-one

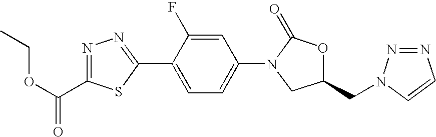

A mixture of (5R)-3-[3-fluoro-4-(trimethylstannyl)phenyl]-5-(1H-1,2,3-triazol-1-ylmethyl)-1,3-oxazolidin-2-one (436 mg, 1.0 mmol), ethyl 5-chloro-1,3,4-thiadiazole-2-carboxylate (197 mg, 0.9 mmol), and tris(2-furyl)phosphine (24 mg, 0.1 mmol) in tetrahydrofuran (5 ml) under an atmosphere of nitrogen was treated with tris(dibenzylideneacetone)dipalladium(0) (47 mg, 0.05 mmol). The mixture was stirred under an atmosphere of nitrogen for 14 hours at 75° C. The solvent was removed under reduced pressure. A solution of the involatile dark oily residue in ethyl acetate (10 ml) was treated with an aqueous solution of potassium fluoride (2M; 10 ml). The mixture was vortexed for two minutes and then stirred for 15 minutes. The mixture was extracted with ethyl acetate (150 ml) and the extract was dried (MgSO₄). The dried extract was filtered, the filtrate was concentrated under reduced pressure, and the residue was purified by chromatography on silica gel (20 g) (dichloromethane to 2% methanol in dichloromethane gradient) to give the desired product (40 mg).

MS (ESP) 419.09 (MH⁺) for C₁₇H₁₅FN₆O₄S ¹H-NMR (DMSO-d₆) δ: 1.31 (t, 3H); 3.93 (dd, 1H); 4.26 (t, 1H); 4.39 (q, 2H); 4.80 (d, 2H); 5.14 (m, 1H); 7.50 (dd, 1H); 7.66 (dd, 1H); 7.70 (s, 1H); 8.12 (s, 1H); 8.32 (t, 1H).

The preparation of ethyl 2-chloro-[1,3,4]thiadiazole-5-carboxylate is described in
Gadwood, Robert C.; Barbachyn, Michael R.; Toops, Dana S.; Smith, Herman W.; Vaillancourt, Valerie A. Preparation of 3-[4-(4-azolyl-1-piperazinyl)phenyl]oxazolidin-2-ones as bactericides. PCT Int. Appl. (1997), 79 pp. CODEN: PIXXD2 WO 9730981 A1 19970828 CAN 127:278210 AN 1997:579705 CAPLUS
Demaree, Patricia; Doria, Marie Carmen; Muchowski, Joseph M. The reaction of certain α-diazocarbonyl compounds with thiophosgene and ethyl chlorodithioformate. Can. J. Chem. (1977), 55(2), 243-50. CODEN: CJCHAG CAN 88:6803 AN 1978:6803 CAPLUS

### EXAMPLE 3

### (5R)-3-(4-(5-(Aminomethyl)-1,3-thiazol-2-yl)-3-fluorophenyl)-5-(1H-1,2,3-triazol-1-ylmethyl)-1,3-oxazolidin-2-one

A mixture of (5R)-3-(3-fluoro-4-(trimethylstannyl)phenyl)-5-(1H-1,2,3-triazol-1-ylmethyl)-1,3-oxazolidin-2-one (425 mg, 1.0 mmol), tert-butyl (2-bromo-1,3-thiazol-5-yl)methylcarbamate (293 mg, 1.0 mmol), copper (I) iodide (38 mg, 0.2 mmol) in DMF (3 ml) under an atmosphere of nitrogen was treated with tetrakis(triphenylphoshine) palladium (0) (56 mg, 0.05 mmol). The mixture was stirred under an atmosphere of nitrogen for 5 hours at 75° C. The reaction mixture was treated with an aqueous solution of potassium fluoride (2M; 10 ml). Ethyl acetate (10 ml) was added and the mixture was vortexed for 5 minutes. The resulting precipitate was filtered off. The filtrate was extracted with ethyl acetate (100 ml) and the extract was dried (MgSO₄). The dried extract was filtered, the filtrate was concentrated under reduced pressure, and the residue was purified by chromatography on silica gel (20 g) (dichloromethane to 10% methanol in dichloromethane gradient) to give a yellow solid. This was dissolved in trifluoroacetic acid (5 ml) and stirred at room temperature for 20 minutes. The trifluoroacetic acid was removed in vacuo and the residue dissolved in DMSO (3 ml). This was purified by reverse phase chromatography (5% acetonitrile to 95% acetonitrile in water) to give the desired product (70 mg).

MS (ESP) 375.11 (MH⁺) for C₁₆H₁₅FN₆O₂S ¹H-NMR (DMSO-d₆) δ: 3.95 (dd, 1H); 4.28 (t, 1H); 4.39 (q, 2H); 4.84 (d, 2H); 5.18 (m, 1H); 7.45 (dd, 1H); 7.63 (dd, 1H); 7.75 (s, 1H); 8.00 (s, 1H); 8.17 (s, 1H); 8.20 (t, 1H); 8.26 (s, 2H).

### EXAMPLE 4

### (5R)-3-(3-Fluoro-4-(5-methyl-1,3,4-thiadiazol-2-yl)phenyl)-5-(1H-1,2,3-triazol-1-ylmethyl)-1,3-oxazolidin-2-one

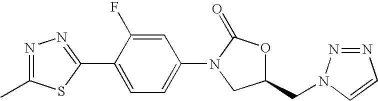

A mixture of 2-fluoro-N′-acetyl-4-((5R)-2-oxo-5-(1H-1,2,3-triazol-1-ylmethyl)-1,3-oxazolidin-3-yl)benzohydrazide (268 mg, 0.74 mmol), and Lawesson's reagent (597 mg, 1.47 mmol) in anhydrous toluene(5 ml) under an atmosphere of nitrogen was heated under reflux overnight. The solvent was removed under reduced pressure and the residue was purified by chromatography on C18 silica gel (Gilson HPLC) (dichloromethane to 2% methanol in dichloromethane gradient) to give the desired product (199 mg).

MS (ESP) 372.06 (MH⁺) for C₁₅H₁₃FN₆O₂S. ¹H-NMR (DMSO-d₆) δ: 2.82 (s, 3H); 3.99 (dd, 1H); 4.33 (t, 1H); 4.89 (d, 2H); 5.21 (m, 1H); 7.52 (m, 1H); 7.69 (dd, 1H); 7.79 (s, 1H); 8.21 (s, 1H); 8.27 (m, 1H).

The intermediates for this compound was prepared as follows:
tert-Butyl 2-fluoro-4-((5R)-5-(1H-1,2,3-triazol-1-ylmethyl)-1,3-oxazolidin-2-on-3-yl)benzoate 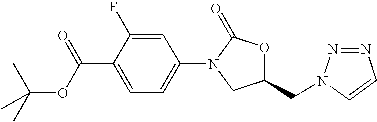
tert-Butyl-4-((5R)-5-(azidomethyl)-1,3-oxazolidin-2-on-3-yl)-2-fluorobenzoate (15 g, 44.6 mmol) was dissolved in dioxane (100 ml). Bicyclo[2.2.1]hepta-2,5-diene (12.3 g, 133.8 mmol) was added and the mixture was heated under reflux under nitrogen for 18 hours. The solvent was evaporated in vacuo and the residue was redissolved in dichloromethane and treated with hexanes to give a precipitate that was filtered, washed with ethyl acetate and collected as the desired product. The filtrate was concentrated and subjected to chromatography on silica gel eluting with 100% ethylacetate to give a further sample of the title compound (combined product weight 14.3 g).

MS (ESP) 363.22 (MH⁺) for C₁₇H₁₉FN₄O₄. ¹H-NMR (DMSO-d₆) δ: 1.55 (s, 9H); 3.97 (m, 1H); 4.28 (t, 1H); 4.86 (d, 2H); 5.20 (m, 1H); 7.39 (dd, 1H); 7.52 (dd, 1H); 7.78 (s, 1H); 7.86 (t, 1H); 8.20 (s, 1H).
tert-Butyl 4-((5R)-5-(azidomethyl)-1,3-oxazolidin-2-on-3-yl)-2-fluorobenzoate 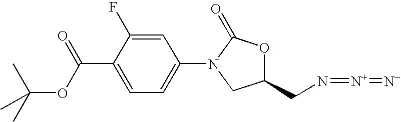
has been previously described in Gordeev, Mikhail F.; Luehr, Gary W.; Patel, Dinesh V.; Gadwood, Robert C. Preparation of N-acyl-3-aryl-2-oxooxazolidine-5-methanamines as bactericides. PCT Int. Appl. (2001), 134 pp. CODEN: PIXXD2 WO 0109107 A1 20010208 CAN 134:163021 AN 2001:101116 CAPLUS
2-Fluoro-4-((5R)-5-(1H-1,2,3-triazol-1-ylmethyl)-1,3-oxazolidin-2-on-3-yl)benzoic acid 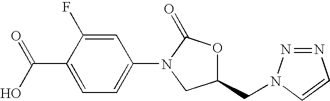
tert-Butyl 2-fluoro-4-((5R)-5-(1H-1,2,3-triazol-1-ylmethyl)-1,3-oxazolidin-2-on-3-yl)benzoate (14.2 g, 39.2 mmol) in dichloromethane was treated with 4N HCl in dioxane (5 equivalent) at 0° C., the mixture was stirred for 2 hours during which it was allowed to warn to room temperature. Solvent was evaporated under reduced pressure to give the desired product (11.9 g) in a form suitable for use without further purification.

MS (ESP) 307.14 (MH⁺) for C₁₃H₁₁FN₄O₄. ¹H-NMR (DMSO-d₆) δ: 3.97 (m, 1H); 4.29 (t, 1H); 4.86 (d, 2H); 5.20 (m, 1H); 7.39 (dd, 1H); 7.51 (dd, 1H); 7.78 (s, 1H); 7.90 (t, 1H); 8.20 (s, 1H); 13.11 (s, bd, 1H).
N′-Acetyl-2-fluoro-4-((5R)-5-(1H-1,2,3-triazol-1-ylmethyl)-1,3-oxazolidin-2-on-3-yl)benzohydrazide

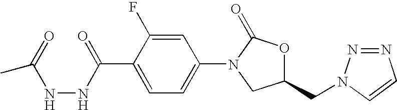

A mixture of 2-fluoro-4-((5R)-5-(1H-1,2,3-triazol-1-ylmethyl)-1,3-oxazolidin-2-on-3-yl)benzoic acid (450 mg, 1.47 mmol), HATU (565 mg, 1.49 mmol) and diisopropylethylamine (285 mg, 2.21 mmol) in dry DMF (5 ml) was stirred at 0° C. for 30 minutes, followed by the addition of acetic acid hydrazide (130.7 mg, 1.76 mmol). The reaction mixture was then warmed up to room temperature and stirred for 2 hours. The mixture was diluted with dichloromethane (20 ml), washed with saturated aqueous NaHCO₃ and brine, dried over anhydrous MgSO₄ and concentrated under reduced pressure. The remaining residue was purified by column chromatography on silica gel (5% MeOH in dichloromethane) to give the desired product (289 mg).

MS (ESP+) 363.16 (MH⁺) for C₁₅H₁₅FN₆O₄. ¹H-NMR (DMSO-d₆) δ: 1.92 (s, 3H); 3.96 (dd, 1H); 4.29 (t, 1H); 4.88 (d, 2H); 5.20 (m, 1H); 7.38 (dd, 1H); 7.53 (dd, 1H); 7.69 (t, 1H); 7.79 (s, 1H); 8.20 (s, 1H); 9.97 (s, 1H); 10.05 (s, 1H).

### EXAMPLE 5

### (5R)-3-(3-Fluoro-4-(4-methyl-1,3-thiazol-2-yl)phenyl)-5-(1H-1,2,3-triazol-1-ylmethyl)-1,3-oxazolidin-2-one

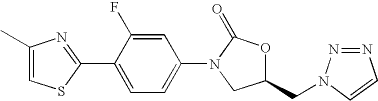

A mixture of 2-fluoro-4-((5R)-5-(1H-1,2,3-triazol-1-ylmethyl)-1,3-oxazolidin-2-on-3-yl)benzenecarbothioamide (30 mg, 0.094 mmol) and 1-chloroacetone (13 mg, 0.14 mmol) in dry DMF was stirred at 60° C. overnight. Solvent was removed under reduced pressure and the residue was purified by reverse phase chromatography (Gilson MPLC C18 column, 5% to 95% acetonitrile in H₂O) to give the title compound (21 mg).

MS (ESP) 360.25 (MH⁺) for C₁₆H₁₄FN₅O₂S. ¹H-NMR (DMSO-d₆) δ: 2.47 (s, 3H); 3.99 (dd, 1H); 4.29 (t, 1H); 4.88 (d, 2H); 5.20 (m, 1H); 7.45 (s, 1H); 7.48 (dd, 1H); 7.64 (dd, 1H); 7.79 (s, 1H); 8.20 (s, 1H); 8.21 (m, 1H).

The intermediates for this compound were prepared as follows:
2-Fluoro-4-((5R)-5-(1H-1,2,3-triazol-1-ylmethyl)-1 3-oxazolidin-2-on-3-yl)benzamide

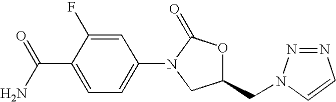

A solution of 2-fluoro-4-((5R)-5-(1H-1,2,3-triazol-1-ylmethyl)-1,3-oxazolidin-2-on-3-yl)benzoic acid (2.5 g, 8.17 mmol) in dry dichloromethane (25 ml) at 0° C. was treated with oxalyl chloride (1.56 g, 12.26 mmol) along with one drop of dimethylformaldehyde. The reaction mixture was allowed to warm to room temperature and then stirred for one hour. Ammonium hydroxide (10 eq.) was then added and the reaction mixture was stirred overnight. The mixture was diluted with additional dichloromethane (20 ml) and then hexanes (30 ml) to give a precipitate that was isolated by filtration to give the desired product (1.5 g).

MS (ESP) 306 (MH⁺) for C₁₃H₁₂FN₅O₃. ¹H-NMR (DMSO-d₆) δ: 3.96 (m, 1H); 4.29 (t, 1H); 4.86 (d, 2H); 5.20 (m, 1H); 7.34 (m, 1H); 7.49 (dd, 1H); 7.60 (s, 2H); 7.74 (t, 1H); 7.78 (s, 1H); 8.20 (s, 1H).
2-Fluoro-4-((5R)-5-(1H-1,2,3-triazol-1-ylmethyl)-1,3-oxazolidin-2-on-3-yl)benzenecarbothioamide

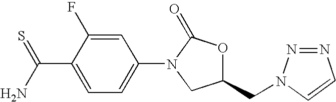

A mixture of 2-fluoro-4-((5R)-5-(1H-1,2,3-triazol-1-ylmethyl)-1,3-oxazolidin-2-on-3-yl)benzamide (95 mg, 0.31 mmol) and Lawesson's reagent (126 mg, 0.31 mmol) in dry toluene (1 ml) was sealed in a microwave reaction vessel and then irradiated in a Smith Microwave reactor at 160° C. for 20 minutes. Solvent was evaporated under reduced pressure and the residue was purified by reverse phase chromatography (Gilson MPLC; C18 column; 5% to 95% acetonitrile in H₂O) to give the title compound (35 mg).

MS (ESP+) 322.24 (MH⁺) for C₁₃H₁₂FN₅O₂S. ¹H-NMR (DMSO-d₆) δ: 3.94 (dd, 1H); 4.28 (t, 1H); 4.87 (d, 2H); 5.19 (m, 1H); 7.28 (dd, 1H); 7.45 (dd, 1H); 7.71 (t, 1H); 7.78 (s, 1H); 8.19 (s, 1H); 9.48 (s, 1H); 10.10 (s, 1H).

### EXAMPLE 6

### (5R)-3-(3-Fluoro-4-(4-(trifluoromethyl)-1,3-thiazol-2-yl)phenyl)-5-(1H-1,2,3-triazol-1-ylmethyl)-1,3-oxazolidin-2-one

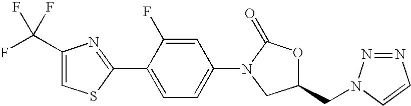 (5R)-3-(3-Fluoro-4-(4-hydroxy-4-(trifluoromethyl)-4,5-dihydro-1,3-thiazol-2-yl)phenyl)-5-(1H-1,2,3-triazol-1-ylmethyl)-1,3-oxazolidin-2-one (25 mg, 0.058 mmol) in ethanol (3 ml) was heated to 150° C. in a sealed tube for 7 days. The solvent was then evaporated and the residue was purified by column chromatography (silica gel; 5% MeOH in dichloromethane) to give the title compound (14 mg).

MS (ESP) 414.18 (MH⁺) for C₁₆H₁₁F4N5O₂S. ¹H-NMR (DMSO-d₆) δ: 3.99 (dd, 1H); 4.29 (t, 1H); 4.88 (d, 2H); 5.20 (m, 1H); 7.45 (dd, 1H); 7.65 (dd, 1H); 7.79 (s, 1H); 8.10 (m, 1H); 8.21 (s, 1H); 8.38 (s, 1H).

The intermediate for this compound was prepared as follows:
(5R)-3-(3-Fluoro-4-(4-hydroxy-4-(trifluoromethyl)-4,5-dihydro-1,3-thiazol-2-yl)phenyl)-5-(1H-1,2,3-triazol-1-ylmethyl)-1,3-oxazolidin-2-one

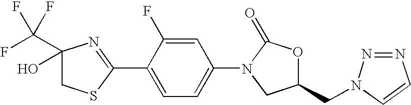

A mixture of 2-fluoro-4-((5R)-5-(1H-1,2,3-triazol-1-ylmethyl)-1,3-oxazolidin-2-on-3-yl)benzenecarbothioamide (30 mg, 0.094 mmol) and 1-bromo-3,3,3-trifluoroacetone (27 mg, 0.14 mmol) in dry DMF was stirred at 60° C. overnight. Solvent was then evaporated under reduced pressure and the residue was purified by reverse phase chromatography (Gilson MPLC; C18 column, 5% to 95% acetonitrile in H₂O) to give the title compound (32 mg).

MS (ESP) 432.07 (MH⁺) for C₁₆H₁₆F₄N₅O₃S. ¹H-NMR (DMSO-d₆) δ: 3.52 (d, 1H); 3.82 (d, 1H); 3.98 (dd, 1H); 4.30 (t, 1H); 4.88 (d, 2H); 5.20 (m, 1H); 7.49 (dd, 1H); 7.62 (dd, 1H); 7.79 (s, 1H); 8.01 (t, 1H); 8.21 (s, 1H).

### EXAMPLE 7

### (5-(2-Fluoro-4-((5R)-5-(1H-1,2,3-triazol-1-ylmethyl)-1,3-oxazolidin-2-on-3-yl)phenyl)-1,3,4-thiadiazol-2-yl)acetonitrile

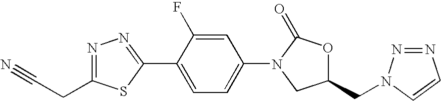

The title compound was prepared from N′-(cyanoacetyl)-2-fluoro-4-((5R)-5-(1H-1,2,3-triazol-1-ylmethyl)-1,3-oxazolidin-2-on-3-yl)benzohydrazide by the method described for Example 4.

MS (ESP+) 386.06 (MH⁺) for C₁₆H₁₂FN₇O₂S. ¹H-NMR (DMSO-d₆) δ: 4.01 (dd, 1H); 4.33 (t, 1H); 4.80 (s, 2H); 4.88 (dd, 2H); 5.20 (m, 1H); 7.58 (dd, 1H); 7.71 (dd, 1H); 7.79 (s, 1H); 8.20 (s, 1H); 8.30 (t, 1H).

The Intermediate for this Compound was Prepared as Follows:
N′-(Cyanoacetyl)-2-fluoro-4-((5R)-5-(1H-1,2,3-triazol-1-ylmethyl)-1,3-oxazolidin-2-on-3-yl)benzohydrazide

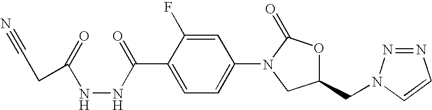

A mixture of 2-fluoro-4-((5R)-5-(1H-1,2,3-triazol-1-ylmethyl)-1,3-oxazolidin-2-on-3-yl)benzoic acid (450 mg, 1.47 mmol), HATU (671 mg, 1.764 mmol) and diisopropylethylamine (285 mg, 2.21 mmol) in dry DMF (5 ml) was stirred at 0° C. for 30 minutes and then treated with cyanoacetohydrazide (219 mg, 2.21 mmol). The reaction mixture was then warmed up to room temperature and stirred for 2 hours. The mixture was diluted with dichloromethane (20 ml), washed with saturated aqueous NaHCO₃ and then brine, dried over anhydrous MgSO₄ and evaporated under reduced pressure. The remaining residue was purified by column chromatography on silica gel (5% MeOH in dichloromethane) to give the desired product (300 mg). ¹H-NMR (DMSO-d₆) δ: 2.71 (s, 2H); 3.96 (dd, 1H); 4.29 (t, 1H); 4.88 (d, 2H); 5.20 (m, 1H); 7.38 (dd, 1H); 7.53 (dd, 1H); 7.69 (t, 1H); 7.79 (s, 1H); 8.20 (s, 1H); 10.32 (s, 1H); 10.44 (s, 1H).

1. A compound of the formula (I), or a pharmaceutically acceptable salt, or an in-vivo-hydrolysable ester thereof, 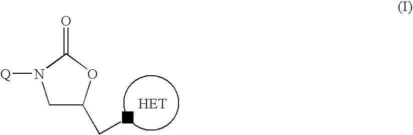
wherein
HET is an N-linked 5-membered, fully or partially unsaturated heterocyclic ring, containing either (i) 1 to 3 further nitrogen heteroatoms or (ii) a further heteroatom selected from O and S together with an optional further nitrogen heteroatom; which ring is optionally substituted on a C atom, other than a C atom adjacent to the linking N atom, with an oxo or thioxo group; and/or which ring is optionally substituted on any available C atom, other than a C atom adjacent to the linking N atom, with a substituent Rs wherein;
Rs is selected from:
(Rsa): halogen, (1-4C)alkoxy, (2-4C)alkenyloxy, (2-4C)alkenyl, (2-4C)alkynyl, (3-6C)cycloalkyl, (3-6C)cycloalkenyl, amino, (1-4C)alkylamino, di-(1-4C)alkylamino, (2-4C)alkenylamino, (1-4C)alkylcarbonylamino, (1-4C)alkylthiocarbonylamino, (1-4C)alkyl-OCO—NH—, (1-4C)alkyl-NH—CO—NH—, (1-4C)alkyl-NH—CS—NH—, (1-4C)alkyl-SO2-NH—, or (1-4C)alkyl-S(O)q- (wherein q is 0, 1, or 2);
(Rsb): (1-4C)alkyl group which is optionally substituted with one substituent selected from hydroxy, (1-4C)alkoxy, amino, cyano, azido, (2-4C)alkenyloxy, (1-4C)alkylcarbonyl, (1-4C)alkoxycarbonyl, (1-4C)alkylamino, (2-4C)alkenylamino, (1-4C)alkyl-SO₂—NH—, (1-4C)alkylcarbonylamino, (1-4C)alkylthiocarbonylamino, (1-4C)alkyl-OCO—NH—, (1-4C)alkyl-NH—CO—NH—, (1-4C)alkyl-NH—CS—NH—, (1-4C)alkyl-SO₂—NH—, (1-4C)alkyl-S(O)q- (wherein q is 0, 1, or 2), (3-6C)cycloalkyl, (3-6C)cycloalkenyl, or an N-linked 5-membered heteroaryl ring, which ring contains either (i) 1 to 3 further nitrogen heteroatoms or (ii) a further heteroatom selected from O and S together with an optional further nitrogen heteroatom; which ring is optionally substituted on a carbon atom with an oxo or thioxo group; and/or the ring is optionally substituted on a carbon atom with 1 or 2 (1-4C)alkyl groups; and/or on an available nitrogen atom (provided that the ring is not thereby quaternised) with (1-4C)alkyl;
(Rsc1):a fully saturated 4-membered monocyclic ring containing 1 or 2 heteroatoms independently selected from O, N, and S (optionally oxidised), and linked via a ring nitrogen or carbon atom;
(Rsc2): a saturated or unsaturated 5-membered monocyclic ring containing 1 heteroatom selected from O, N, and S (optionally oxidised), and linked via a ring nitrogen atom if the ring is not thereby quaternised, or a ring carbon atom;
(Rsc3): a saturated or unsaturated 6- to 8-membered monocyclic ring containing 1 or 2 heteroatoms independently selected from O, N, and S (optionally oxidised), and linked via a ring nitrogen atom if the ring is not thereby quaternised, or a ring carbon atom;
wherein said rings in (Rsc1) to (Rsc3) are optionally substituted on an available carbon atom with 1 or 2 substituents independently selected from hydroxy, (1-4C)alkoxy, amino, cyano, azido, (2-4C)alkenyloxy, (1-4C)alkylcarbonyl, (1-4C)alkoxycarbonyl, (1-4C)alkylamino, (2-4C)alkenylamino, (1-4C)alkyl-SO₂—NH—, (1-4C)alkylcarbonylamino, (1-4C)alkylthiocarbonylamino, (1-4C)alkyl-OCO—NH—, (1-4C)alkyl-NH—CO—NH—, (1-4C)alkyl-NH—CS—NH—, (1-4C)alkyl-SO₂—NH—, (1-4C)alkyl-S(O)q- (wherein q is 0, 1, or 2), (3-6C)cycloalkyl, or (3-6C)cycloalkenyl;
(Rsd): cyano, nitro, azido, formyl, (1-4C)alkylcarbonyl, or (1-4C)alkoxycarbonyl;
wherein at each occurrence of an Rs substituent containing an alkyl, alkenyl, alkynyl, cycloalkyl, or cycloalkenyl moiety in (Rsa), (Rsb) or (Rsc1) to (Rsc3) each such moiety is optionally further substituted on an available carbon atom with one or more substituents independently selected from F, Cl, and Br and/or with one cyano group; and/or which ring is optionally substituted on an available nitrogen atom (provided that the ring is not thereby quaternised) with (1-4C)alkyl; or
HET is an N-linked 6-membered dihydro-heteroaryl ring containing up to three nitrogen heteroatoms in total (including the linking heteroatom), which ring is substituted on a suitable C atom, other than a C atom adjacent to the linking N atom, with oxo or thioxo and/or which ring is optionally substituted on any available C atom, other than a C atom adjacent to the linking N atom, with one or two substituents Rs, wherein Rs is as hereinbefore defined, and/or on an available nitrogen atom (provided that the ring is not thereby quaternised) with (1-4C)alkyl; and wherein at each occurrence of alkyl, alkenyl, and cycloalkyl HET substituents, each is optionally substituted with one or more substituents independently selected from F, Cl, and Br and/or with one cyano group;
Q is selected from Q1 to Q10: 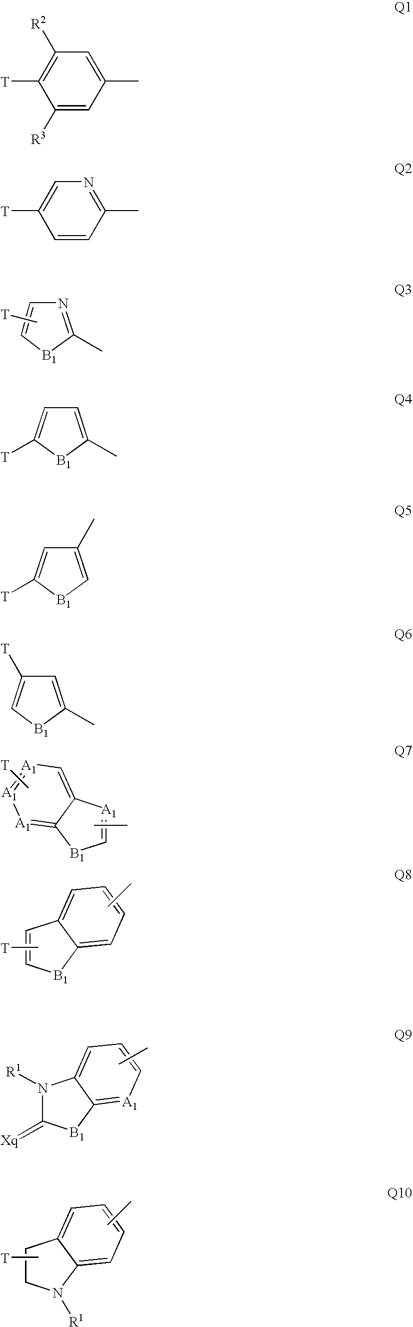
wherein R² and R³ are independently hydrogen or fluoro;
A₁ is carbon or nitrogen;
B₁ is O or S (or, in Q9 only, NH);
X_{q} is O, S, or N-R¹ (wherein R¹ is hydrogen, (1-4C)alkyl, or hydroxy-(1-4C)alkyl); and
in Q7 each A₁ is independently selected from carbon or nitrogen, with a maximum of 2 nitrogen heteroatoms in the 6-membered ring, and Q7 is linked to T via any of the A₁ atoms (when A₁ is carbon), and linked in the 5-membered ring via the specified carbon atom, or via A₁ when A₁ is carbon; Q8 and Q10 are linked to T via either of the specified carbon atoms in the 5-membered ring, and linked to the benzo-ring via either of the two specified carbon atoms on either side of the linking bond shown; and Q9 is linked via either of the two specified carbon atoms on either side of the linking bond shown;
T is an optionally substituted C-linked (fully unsaturated) 5-membered heteroaryl ring system containing 1, 2, or 3 heteroatoms selected from O, N, or S, optionally substituted with one or more substituents independently selected from R^{4h}, R^{5h}, and R^{6h} defined hereinafter; or
T is selected from the following groups of formula (TAa1) to (TAa6) below (wherein AR1, AR2, AR2a, AR2b, AR3, AR3a, AR3b, AR4, AR4a, CY1, and CY2 are defined hereinbelow); 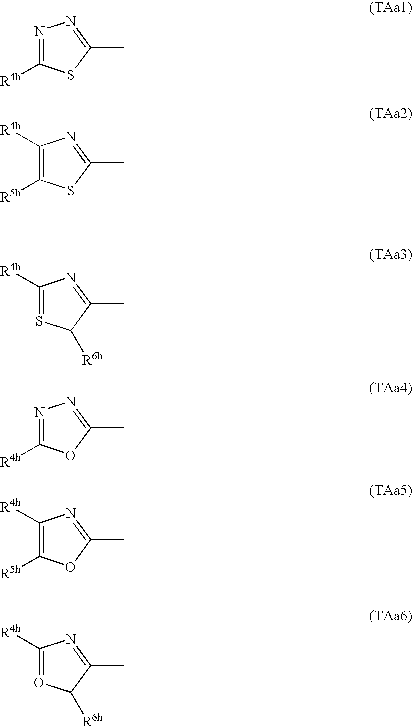
wherein R^{6h} is selected from hydrogen, (1-4C)alkyl, (1-4C)alkoxycarbonyl, (1-4C)alkanoyl, carbamoyl, and cyano;
R^{4h} and R^{5h} are independently selected from hydrogen, halo, trifluoromethyl, cyano, nitro, (1-4C)alkoxy, (1-4C)alkylS(O)_{q}— (q is 0, 1, or 2), (1-4C)alkanoyl, (1-4C)alkoxycarbonyl, benzyloxy-(1-4C)alkyl, (2-4C)alkanoylamino, —CONRcRv, and —NRcRv, wherein any (1-4C)alkyl group contained in the preceding values for R^{4h} and R^{5h} is optionally substituted with up to three substituents independently selected from hydroxy (not on C1 of an alkoxy group, and excluding geminal disubstitution), oxo, trifluoromethyl, cyano, nitro, (1-4C)alkoxy, (2-4C)alkanoyloxy, hydroxyimino, (1-4C)alkoxyimino, (1-4C)alkylS(O)_{q}— (q is 0, 1, or 2), (1-4C)alkylSO₂—NRv-, (1-4C)alkoxycarbonyl, —CONRcRv, and —NRcRv (not on C1 of an alkoxy group, and excluding geminal disubstitution); wherein Rv is hydrogen or (1-4C)alkyl and Rc is as hereinafter defined;
R^{4h} and R^{5h} may further be independently selected from (1-4C)alkyl {optionally substituted with up to three substituents independently selected from hydroxy (excluding geminal disubstitution), oxo, trifluoromethyl, cyano, nitro, (1-4C)alkoxy, (2-4C)alkanoyloxy, hydroxyimino, (1-4C)alkoxyimino, (1-4C)alkylS(O)_{q}— (q is 0, 1, or 2), (1-4C)alkylSO₂—NRv-, (1-4C)alkoxycarbonyl, —CONRcRv, and —NRcRv (excluding geminal disubstitution); wherein Rv is hydrogen or (1-4C)alkyl}; Rc is as hereinafter defined; and any (1-4C)alkyl group contained in the immediately preceding optional substituents (when R^{4h} and R^{5h} are independently (1-4C)alkyl) is itself optionally substituted with up to three substituents independently selected from hydroxy (not on C1 of an alkoxy group, and excluding geminal disubstitution), oxo, trifluoromethyl, cyano, nitro, (1-4C)alkoxy, (2-4C)alkanoyloxy, hydroxyimino, (1-4C)alkoxyimino, (1-4C)alkylS(O)_{q}— (q is 0, 1, or 2), (1-4C)alkylSO₂—NRv-, (1-4C)alkoxycarbonyl, —CONRcRv, and —NRcRv (not on C1 of an alkoxy group, and excluding geminal disubstitution); wherein Rv is hydrogen or (1-4C)alkyl, and Rc is as hereinafter defined; or
R^{4h} is selected from one of the groups in (TAaa) to (TAac) below, or (where appropriate) one of R^{4h} and R^{5h} is selected from the above list of R^{4h} and R^{5h} values, and the other is selected from one of the groups in (TAaa) to (TAac) below:
(TAaa)a group of the formula (TAaa1) 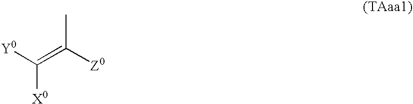
wherein Z⁰ is hydrogen or (1-4C)alkyl;
X⁰ and Y⁰ are independently selected from hydrogen, (1-4C)alkyl, (1-4C)alkoxycarbonyl, halo, cyano, nitro, (1-4C)alkylS(O)_{q}— (q is 0, 1, or 2), RvRwNSO₂—, trifluoromethyl, pentafluoroethyl, (1-4C)alkanoyl, and —CONRvRw [wherein Rv is hydrogen or (1-4C)alkyl; and Rw is hydrogen or (1-4C)alkyl]; or
one of X⁰ and Y⁰ is selected from the above list of X⁰ and Y⁰ values, and the other is selected from phenyl, phenylcarbonyl, —S(O)_{q}-phenyl (q is 0, 1, or 2), N-(phenyl)carbamoyl, phenylaminosulfonyl, AR2, (AR2)-CO—, (AR2)-S(O)q- (q is 0, 1, or 2), N-(AR2)carbamoyl, and (AR2)aminosulfonyl; wherein any phenyl group in (TAaa) may be optionally substituted with up to three substituents independently selected from (1-4C)alkyl, cyano, trifluoromethyl, nitro, halo, and (1-4C)alkylsulfonyl;
(TAab) an acetylene of the formula —≡—H or -≡-(1-4C)alkyl;
(TAac) —X¹—Y¹-AR2, —X¹—Y¹-AR2a, —X¹—Y¹-AR2b, —X¹—Y¹-AR3, —X¹—Y¹-AR3a, or —X¹—Y¹-AR3b;
wherein X¹ is a direct bond or —CH(OH)— and Y¹ is —(CH₂)ₘ—, —(CH₂)ₙ—NH—(CH₂)ₘ—, —CO—(CH₂)ₘ—, —CONH—(CH₂)ₘ—, —C(═S)NH—(CH₂)ₘ—, or —C(═O)O—(CH₂)ₘ—, —(CH₂)ₙ— or —CH(Me)-(CH₂)ₘ— and Y¹ is —(CH₂)ₘ—NH—(CH₂)ₘ—, —CO—(CH₂)ₘ—, —CONH—(CH₂)ₘ—, —C(═S)NH—(CH₂)ₘ—, —C(═O)O—(CH₂)ₘ—, —S(O)_{q}—(CH2)ₘ—, —CH₂O—, —CH₂NH— or —CH₂N((1-4C)alkyl)- and Y¹ is —CO—(CH₂)ₘ—, —CONH—(CH₂)ₘ—, or —C(═S)NH—(CH₂)ₘ—;
Y¹ is —SO₂— when X¹ is —CH₂NH— or —CH₂N((1-4C)alkyl)-, and Y¹ is —(CH₂)ₘ— when X¹ is —CH₂O— or —CH₂N((1-4C)alkyl)-;
n is 1, 2, or 3; m is 0, 1, 2, or 3 and q is 0, 1, or 2; and when Y¹ is —(CH₂)ₘ—NH—(CH₂)ₘ— each m is independently selected from 0, 1, 2, or 3;
wherein Rc is selected from groups (Rc 1) to (Rc5):
(Rc1) (1-6C)alkyl {optionally substituted with one or more (1-4C)alkanoyl groups (including geminal disubstitution) and/or optionally monosubstituted with cyano, (1-4C)alkoxy, trifluoromethyl, (1-4C)alkoxycarbonyl, phenyl (optionally substituted as for AR1 defined hereinafter), (1-4C)alkylS(O)_{q}— (q is 0, 1, or 2); or on any but the first carbon atom of the (1-6C)alkyl chain, optionally substituted with one or more groups (including geminal disubstitution) each independently selected from hydroxy and fluoro, and/or optionally monosubstituted with oxo, —NRvRw [wherein Rv is hydrogen or (1-4C)alkyl; Rw is hydrogen or (1-4C)alkyl], (1-6C)alkanoylamino, (1-4C)alkoxycarbonylamino, N-(1-4C)alkyl-N-(1-6C)alkanoylamino, (1-4C)alkylS(O)ₚNH—, or (1-4C)alkylS(O)ₚ-((1-4C)alkyl)N— (p is 1 or 2)};
(Rc2) R¹³CO—, R¹³SO₂—, or R¹³CS—;
wherein R¹³ is selected from (Rc2a) to (Rc2e)
(Rc2a) AR1, AR2, AR2a, AR2b, AR3, AR3a, AR3b, AR4, AR4a, CY1, or CY2;
(Rc2b) hydrogen, (1-4C)alkoxycarbonyl, trifluoromethyl, —NRvRw [wherein Rv is hydrogen or (1-4C)alkyl; Rw is hydrogen or (1-4C)alkyl], ethenyl, 2-(1-4C)alkylethenyl, 2-cyanoethenyl, 2-cyano-2-((1-4C)alkyl)ethenyl, 2-nitroethenyl, 2-nitro-2-((1-4C)alkyl)ethenyl, 2-((1-4C)alkylaminocarbonyl)ethenyl, 2-((1-4C)alkoxycarbonyl)ethenyl, 2-(AR1)ethenyl, 2-(AR2)ethenyl, or 2-(AR2a)ethenyl;
(Rc2c) (1-10C)alkyl {optionally substituted with one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkanoyl, carboxy, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted with one group selected from phosphonate [phosphono, —P(O)(OH)₂, and mono- and di-(1-4C)alkoxy derivatives thereof], phosphinate [—P(OH)₂ and mono- and di-(1-4C)alkoxy derivatives thereof], cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino, (1-4C)alkoxycarbonylamino, N-(1-4C)alkyl-N-(1-6C)alkanoylamino, (1-4C)alkylaminocarbonyl, di((1-4C)alkyl)aminocarbonyl, (1-4C)alkylS(O)ₚNH—, (1-4C)alkylS(O)ₚ-((1-4C)alkyl)N—, fluoro(1-4C)alkylS(O)ₚNH—, fluoro(1-4C)alkylS(O)ₚ((1-4C)alkyl)N—, (1-4C)alkylS(O)_{q}— [the (1-4C)alkyl group of (1-4C)alkylS(O)_{q}— being optionally substituted with one substituent selected from hydroxy, (1-4C)alkoxy, (1-4C)alkanoyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], amino, cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, carboxy, (1-4C)alkylamino, di((1-4C)alkyl)amino, (1-6C)alkanoylamino, (1-4C)alkoxycarbonylamino, N-(1-4C)alkyl-N-(1-6C)alkanoylamino, (1-4C)alkylaminocarbonyl, di((1-4C)alkyl)aminocarbonyl, (1-4C)alkylS(O)ₚNH—, (1-4C)alkylS(O)ₚ-((1-4C)alkyl)N—, (1-4C)alkylS(O)_{q}—, AR1-S(O)_{q}—, AR2-S(O)_{q}—, AR3-S(O)_{q}—, and AR2a, AR2b, AR3a, and AR3b versions of AR2 and AR3 containing groups], CY1, CY2, AR1, AR2, or AR3,
AR1-O—, AR2-O—, AR3-O—, AR1-S(O)_{q}—, AR2-S(O)_{q}—, AR3-S(O)_{q}—, AR1-NH—, AR2-NH—, AR3-NH— (p is 1 or 2 and q is 0, 1 or 2), and AR2a, AR2b, AR3a, and AR3b versions of AR2 and AR3 containing groups};
(Rc2d) R¹⁴C(O)O(1-6C)alkyl wherein R¹⁴ is AR1, AR2, (1-4C)alkylamino (the
(1-4C)alkyl group being optionally substituted with (1-4C)alkoxycarbonyl, carboxy), benzyloxy-(1-4C)alkyl, or (1-10C)alkyl {optionally substituted as defined for (Rc2c)};
(Rc2e) R¹⁵O— wherein R¹⁵ is benzyl, (1-6C)alkyl {optionally substituted as defined for (Rc2c)}, CY1, CY2, or AR2b;
(Rc3) hydrogen, cyano, 2-cyanoethenyl, 2-cyano-2-((1-4C)alkyl)ethenyl, 2-((1-4C)alkylaminocarbonyl)ethenyl, 2-((1-4C)alkoxycarbonyl)ethenyl, 2-nitroethenyl, 2-nitro-2-((1-4C)alkyl)ethenyl, 2-(AR1)ethenyl, 2-(AR2)ethenyl, or of the formula (Rc3a) 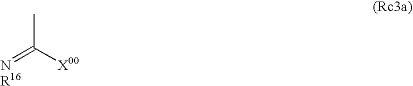
wherein X⁰⁰ is —OR¹⁷, —SR¹⁷, —NHR¹⁷, or —N(R¹⁷)₂;
R¹⁷ is hydrogen when X⁰⁰ is —NHR¹⁷ or —N(R¹⁷)₂, R¹⁷ is (1-4C)alkyl, phenyl, or AR2 when X⁰⁰ is —OR¹⁷, —SR¹⁷, or —NHR¹⁷; and
R¹⁶ is cyano, nitro, (1-4C)alkylsulfonyl, (4-7C)cycloalkylsulfonyl, phenylsulfonyl, (1-4C)alkanoyl, or (1-4C)alkoxycarbonyl;
(Rc4) trityl, AR1, AR2, AR2a, AR2b, AR3, AR3a, or AR3b;
(Rc5) RdOC(Re)=CH(C═O)—, RfC(═O)C(═O)—, RgN═C(Rh)C(═O)—, or RiNHC(Rj)=CHC(═O)— wherein Rd is (1-6C)alkyl; Re is hydrogen or (1-6C)alkyl, or Rd and Re together form a (3-4C)alkylene chain; Rf is hydrogen, (1-6C)alkyl, hydroxy(1-6C)alkyl, (1-6C)alkoxy(1-6C)alkyl, —NRvRw [wherein Rv is hydrogen or (1-4C)alkyl; Rw is hydrogen or (1-4C)alkyl], (1-6C)alkoxy, (1-6C)alkoxy(1-6C)alkoxy, hydroxy(2-6C)alkoxy, (1-4C)alkylamino(2-6C)alkoxy, di-(1-4C)alkylamino(2-6C)alkoxy; Rg is (1-6C)alkyl, hydroxy, or (1-6C)alkoxy; Rh is hydrogen or (1-6C)alkyl; Ri is hydrogen, (1-6C)alkyl, AR1, AR2, AR2a, AR2b; and Rj is hydrogen or (1-6C)alkyl;
wherein
AR1 is an optionally substituted phenyl or optionally substituted naphthyl;
AR2 is an optionally substituted 5- or 6-membered, fully unsaturated monocyclic heteroaryl ring containing up to four heteroatoms independently selected from O, N, and S (but not containing any O—O, O—S or S—S bonds), and linked via a ring carbon atom, or a ring nitrogen atom if the ring is not thereby quaternised;
AR2a is a partially hydrogenated version of AR2 linked via a ring carbon atom or linked via a ring nitrogen atom if the ring is not thereby quaternised;
AR2b is a fully hydrogenated version of AR2 linked via a ring carbon atom or linked via a ring nitrogen atom;
AR3 is an optionally substituted 8-, 9-, or 10-membered, fully unsaturated bicyclic heteroaryl ring containing up to four heteroatoms independently selected from O, N, and S (but not containing any O—O, O—S or S—S bonds), and linked via a ring carbon atom in either of the rings comprising the bicyclic system;
AR3a is a partially hydrogenated version of AR3 linked via a ring carbon atom, or linked via a ring nitrogen atom if the ring is not thereby quaternised, in either of the rings comprising the bicyclic system;
AR3b is a fully hydrogenated version of AR3, linked via a ring carbon atom, or linked via a ring nitrogen atom, in either of the rings comprising the bicyclic system;
AR4 is an optionally substituted 13- or 14-membered, fully unsaturated tricyclic heteroaryl ring containing up to four heteroatoms independently selected from O, N, and S (but not containing any O—O, O—S or S—S bonds), and linked via a ring carbon atom in any of the rings comprising the tricyclic system;
AR4a is a partially hydrogenated version of AR4, linked via a ring carbon atom, or linked via a ring nitrogen atom if the ring is not thereby quaternised, in any of the rings comprising the tricyclic system;
CY1 is an optionally substituted cyclobutyl, cyclopentyl, or cyclohexyl ring;
CY2 is an optionally substituted cyclopentenyl or cyclohexenyl ring.

2. A compound of claim 1, or a pharmaceutically acceptable salt, or in-vivo hydrolysable ester thereof, wherein Q is selected from Q1, Q2, Q4, Q6, and Q9.

3. A compound of claim 1, or a pharmaceutically acceptable salt, or in-vivo hydrolysable ester thereof, wherein T is selected from (TAa1) to (TAa6).

4. A compound of formula (IB), or a pharmaceutically acceptable salt, or in-vivo hydrolysable ester thereof, 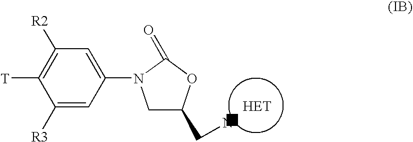
wherein HET is 1,2,3-triazole, 1,2,4-triazole, or tetrazole, or HET is a dihydro version of pyrimidine, pyridazine, pyrazine, 1,2,3-triazine, 1,2,4-triazine, 1,3,5-triazine, or pyridine;
R² and R³ are independently hydrogen or fluoro; and
T is selected from (TAa1 to TAa6).

5. A compound of claim 4, or a pharmaceutically acceptable salt, or in-vivo hydrolysable ester thereof, wherein
HET is 1,2,3-triazole, 1,2,4-triazole, or tetrazole;
R² and R³ are independently hydrogen or fluoro; and
T is selected from (TAa1 and 2).

6. A compound of the formula (I) as claimed in any preceding claim, or a pharmaceutically acceptable salt, or in-vivo hydrolysable ester thereof, wherein Rs is selected from fluoromethyl, difluoromethyl, trifluoromethyl, chloromethyl, bromomethyl, cyanomethyl, cyano, amino, azido, alkylthioalkyl, and 2-propynyl.

7. A pharmaceutical composition which comprises a compound of claim 1, or a pharmaceutically acceptable salt or an in-vivo hydrolysable ester thereof, and a pharmaceutically acceptable diluent or carrier.

8. A method for producing an antibacterial effect in a warm blooded animal in need of such treatment, which comprises administering to said animal an effective amount of a compound of a compound of claim 1, or a pharmaceutically acceptable salt, or an in-vivo hydrolysable ester thereof.

9. A process for the manufacture of a compound of claim 1, comprising one or more of the processes (a) to (i) below:
(a) modifying a substituent in or introducing a substituent into another compound of formula (I);
(b) reaction of a compound of formula (II): 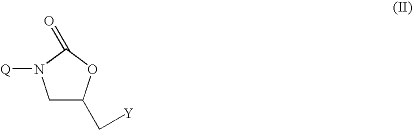
wherein Y is a displaceable group with a compound of the formula (III):
HET (III)
wherein HET is HET-H free-base form or HET-anion formed from the free base form;
(c) reaction of a compound of the formula (IV):
Q-Z (IV)
wherein Z is an isocyanate, amine or urethane group with an epoxide of the formula (V): 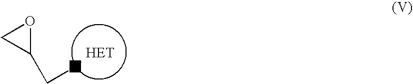
(d) reaction of a compound of formula (VI): 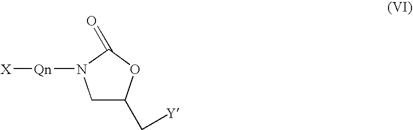
wherein Y′ is a group HET as hereinabove defined, X is a replaceable substituent located at a position substituted by T in any of the aromatic embodiments Q1-Q8 of Qn as hereinabove defined for Q, but with X in place of the substituent T, with a compound of the formula (VII):
T-X′ (VII)
wherein T-X′ is a five-membered heterocycle with 1-3 heteroatoms drawn in combination from O, N, and S and X′ is a replaceable C-linked substituent; wherein the substituents X and X′ are chosen to be complementary pairs of substituents known in the art to be suitable as complementary substrates for coupling reactions catalysed by transition metals such as palladium(0);
(e) reaction of a compound of formula (VIII): 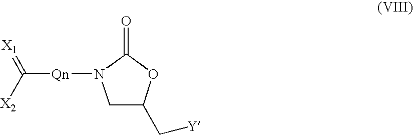
wherein Y′ is a group HET as defined herein above and X1 and X2 here are independently optionally substituted heteroatoms drawn in combination from O, N, and S such that C(X1)X2 constitutes a substituent that is a carboxylic acid derivative located at a position substituted by T in any of the aromatic embodiments Q1-Q10 of Qn as hereinabove defined for Q with a compound of the formula (IX) and X3 and X4 are independently optionally substituted heteroatoms drawn in combination from O, N, and S: 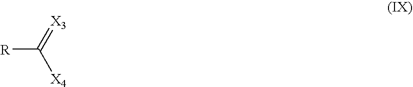
and one of C(X1)X2 and C(X3)X4 constitutes an optionally substituted hydrazide, thiohydrazide, or amidrazone, and the other one of C(X1)X2 and C(X3)X4 constitutes an optionally substituted acylating, thioacylating, or imidoylating agent such that C(X1)X2 and C(X3)X4 may be condensed together to form a 5-membered heterocycle containing 3 heteroatoms drawn in combination from O, N, and S;
(f) reaction of a compound of formula (X): 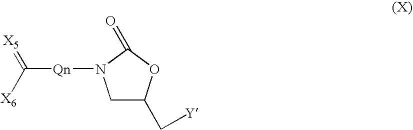
wherein Y′ is a group HET as defined herein above and C(X5)X6 constitutes a substituent located at a position substituted by T in any of the aromatic embodiments Q1-Q8 of Qn as hereinabove defined for Q with a compound of the formula (XI): 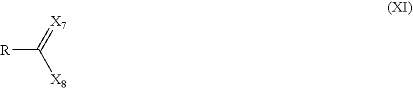
wherein one of C(X5)X6 and C(X7)X8 constitutes an optionally substituted alpha-(leaving-group-substituted)ketone, and the other one of C(X5)X6 and C(X7)X8 constitutes an optionally substituted amide, thioamide, or amidine, such that C(X5)X6 and C(X7)X8 are groups that may be condensed together to form a 5-membered heterocycle containing 2 heteroatoms drawn in combination from O, N, and S, by methods well-known in the art;
(g) for HET as optionally substituted 1,2,3-triazoles compounds of formula (I), cycloaddition via the azide to acetylenes, or to acetylene equivalents;
(h) for HET as 4-substituted 1,2,3-triazole compounds of formula (I), reacting aminomethylisoxazolines with 1,1-dihaloketone sulfonylhydrazones;
(i) for HET as 4-substituted 1,2,3-triazole compounds of formula (I), reacting azidomethyl isoxazolines with terminal alkynes using Cu(I) catalysis;
and thereafter if necessary: (i) removing any protecting groups; (ii) forming a pharmaceutically acceptable salt; or (iii) forming an in-vivo hydrolysable ester.

10. A compound selected from
(5R)-3-(3-Fluoro-4-(5-cyano-1,3,4-thiadiazol-2-yl)phenyl)-5-(1H-1,2,3-triazol-1-ylmethyl)-1,3-oxazolidin-2-one;
(5R)-3-(3-Fluoro-4-(5-ethoxycarbonyl-1,3,4-thiadiazol-2-yl)phenyl)-5-(1H-1,2,3-triazol-1-ylmethyl)-1,3-oxazolidin-2-one;
(5R)-3-(4-(5-(Aminomethyl)-1,3-thiazol-2-yl)-3-fluorophenyl)5-(1H-1,2,3-triazol-1-ylmethyl)-1,3-oxazolidin-2-one;
(5R)-3-(3-Fluoro-4-(5-methyl-1,3,4-thiadiazol-2-yl)phenyl)-5-(1H-1,2,3-triazol-1-ylmethyl)-1,3-oxazolidin-2-one;
(5R)-3-(3-Fluoro-4-(4-methyl-1,3-thiazol-2-yl)phenyl)-5-(1H-1,2,3-triazol-1-ylmethyl)-1,3-oxazolidin-2-one;
(5R)-3-(3-Fluoro-4-(4-(trifluoromethyl)-1,3-thiazol-2-yl)phenyl)-5-(1H-1,2,3-triazol-1-ylmethyl)-1,3-oxazolidin-2-one; and
(5-(2-Fluoro-4-((5R)-5-(1H-1,2,3-triazol-1-ylmethyl)-1,3-oxazolidin-2-on-3-yl)phenyl)-1,3,4-thiadiazol-2-yl)acetonitrile;
or a pharmaceutically acceptable salt or an in-vivo hydrolysable ester thereof.
